(19) United States
(12) Patent Application Publication
(10) Pub. No.: US 2002/0048550 A1
(43) Pub. Date: Apr. 25, 2002

(54) Radiolabeled immunotoxins

(76) Inventors: Daniel A. Vallera, St. Louis Park, MN (US); Donald J. Buchsbaum, Birmingham, AL (US)

Correspondence Address: MARK S. ELLINGER, PH.D., Fish & Richardson P.C., Suite 2800, 45 Rockefeller Plaza, New York, NY, 10111, US

(21) Appl. No.: 09/910,639

(22) Filed: Jul. 20, 2001

Related U.S. Application Data
(60) Provisional application No. 60/219,759, filed on Jul. 20, 2000.

(51) Int. Cl. A61K 51/00; C12N 9/22; C07K 14/415; C07K 14/24; C07K 14/00

(52) U.S. Cl. 424/1.69; 424/1.57; 530/350; 435/199; 530/370

(57) ABSTRACT

The invention features radiolabeled immunotoxins, and radiolabeled multimeric (e.g., dimeric) immunotoxins. Also encompassed by the invention are methods of killing pathogenic cells, imaging, and making radiolabeled immunotoxins and radiolabeled multimeric immunotoxins.

This application claims priority of U.S. provisional application Ser. No. 60/219,759, filed Jul. 20, 2000.

Some of the research described in this application was supported by a grant (no. DE-FG02-96ER62181) from the U.S. Department of Energy. The U.S. government may have certain rights in the invention.

### BACKGROUND OF THE INVENTION

The invention is generally in the field of immunotoxins, particularly radiolabeled immunotoxins effective against pathogenic cells, e.g., breast, brain, ovarian or colon cancer cells.

Immunotoxins are molecules that contain targeting domains that direct the molecules to target cells of interest (e.g., cancer cells or effector T lymphocytes) and toxic domains that kill the target cells. They are thus useful in the treatment of pathological conditions such as cancer, graft-versus-host disease (GVHD), autoimmune diseases, and certain infectious diseases.

### SUMMARY OF THE INVENTION

The invention derives from the finding that radiolabeled immunotoxins (RIT) substantially retained the cytotoxic activity of the corresponding unlabeled immunotoxin (IT) and showed greater cytotoxic activity in vivo than the unlabeled IT. The invention includes RIT, and radiolabeled multimeric (e.g., dimeric) IT (RMIT). Also encompassed by the invention are in vitro and in vivo methods of killing a target cell using the RIT and RMIT and methods of producing the RIT and RMIT.

More specifically, the invention features a radiolabeled immunotoxin that includes a toxic domain, a targeting domain, and at least one radionuclide atom. The targeting domain can be, for example, a single-chain Fv antibody fragment that binds to a target molecule on a target cell, with the target molecule preferably not being a polypeptide of the T cell CD3 complex. In a more preferred embodiment the target molecule is not the ε chain of the T cell CD3 complex. In the radiolabeled immunotoxin of the invention, the toxic domain can be a toxic polypeptide, e.g., (a) ricin, (b) Pseudomonas exotoxin (PE); (c) bryodin; (d) gelonin; (e) α-sarcin; (f) aspergillin; (g) restrictocin; (h) angiogenin; (i) saporin; (j) abrin; (k)pokeweed antiviral protein (PAP); (1) a ribonuclease; (m) a pro-apoptotic polypeptide, or (n) a functional fragment of any of (a)-(m). The toxic domain can also be diphtheria toxin (DT) or a functional fragment thereof, e.g., amino acids 1-389 of DT. The target cell of the radiolabeled immunotoxin can be a cancer cell (e.g., a neural tissue cancer cell, a melanoma cell, a breast cancer cell, a lung cancer cell, a gastrointestinal cancer cell, an ovarian cancer cell, a testicular cancer cell, a lung cancer cell, a prostate cancer cell, a cervical cancer cell, a bladder cancer cell, a vaginal cancer cell, a liver cancer cell, a renal cancer cell, a bone cancer cell, or a vascular tissue cancer cell) and the target molecule can be Her-2/neu, a mucin molecule, carcinoembryonic antigen (CEA), prostate-specific antigen (PSA), folate binding receptor, A33 alpha fetoprotein, CA-125 glycoprotein, colon-specific antigen p, ferritin, p-glycoprotein, G250, OA3, PEM glycoprotein, L6 antigen, 19-9 P97, placental alkaline phosphatase, 7E11-C5, 17-1A, TAG-72, 40 kDa glycoprotein, URO-8, a tyrosinase, an interleukin- (IL- )2 receptor polypeptide, an IL-3 receptor polypeptide, an IL-13 receptor polypeptide, an IL-4 receptor polypeptide, a vascular endothelial growth factor (VEGF) receptor, a granulocyte macrophage-colony stimulating factor (GM-CSF) receptor polypeptide, an epidermal growth factor (EGF) receptor polypeptide, an insulin receptor polypeptide, an insulin-like growth factor receptor polypeptide, transferrin receptor, estrogen receptor, a T cell receptor (TCR) α-chain, a TCR β-chain, a CD4 polypeptide, a CD8 polypeptide, a CD7 polypeptide, a B cell immunoglobulin (Ig) heavy chain, a B cell Ig light chain, a CD19 polypeptide, a CD20 polypeptide, a CD22 polypeptide, a MAGE polypeptide, a BAGE polypeptide, a GAGE polypeptide, a RAGE polypeptide, a PRAME polypeptide, or a GnTV polypeptide. The radionuclide can be, for example, ⁹⁰Y, ¹⁸⁶Re, ¹⁸⁸Re, ⁶⁴Cu, ⁶⁷Cu, ²¹²Pb, ²¹²Bi, ²¹³Bi, ¹²³I, ¹²⁵I, ¹³¹I, ²¹¹At, ³²P, ¹⁷⁷Lu, ⁴⁷Sc, ¹⁰⁵Rh, ¹⁰⁹Pd, ¹⁵³Sm, ¹⁹⁹Au, ^{99m}Tc, ¹¹¹In, ¹²⁴I, ¹⁸F, ¹¹C, ¹⁹⁸Au, ⁷⁵Br, ⁷⁶Br, ⁷⁷Br, ¹³N, ^{34m}Cl, ³⁸Cl, ^{52m}Mn, ⁵⁵Co, ⁶²Cu, ⁶⁸Ga, ⁷²As, ⁷⁶As, ⁷²Se, ⁷³Se, and ⁷⁵Se.

Also encompassed by the invention is a radiolabeled multimeric (e.g., dimeric) immunotoxin that includes: (a) at least two monomers, and (b) at least one radionuclide atom. Each monomer of the radiolabeled multimeric immunotoxin contains a targeting domain and a toxic domain and is physically associated with the other monomers and the targeting domain can bind to a target molecule on a target cell. Each of the monomers can further comprise one or more coupling moieties and the physical association of the monomer is by at least one of the one or more coupling moieties, e.g., a terminal moiety (i.e., a C terminal or an N-terminal moiety). The one or more coupling moieties can be cysteine residues and can be heterologous coupling moieties. In the radiolabeled multimeric immunotoxin, each of the monomers can have the same amino acid sequence or a different amino acid sequence. The targeting domain can be an antibody fragment, e.g., a sFv. The antibody fragment can bind to a target molecule on a T cell (e.g., a CD3 complex polypeptide) or a cancer cell, e.g., any of the cancer cells listed above. In the radiolabeled multimeric immunotoxins, the targeting domain can be a targeting polypeptide, e.g., (a) a cytokine; (b) a ligand for a cell adhesion receptor; (c) a ligand for a signal transduction receptor; (d) a hormone; (e) a molecule that binds to a death domain family molecule; (f) an antigen; and (g) a functional fragment of any of (a) - (f).

The invention also features an in vitro method of killing a target cell. The method involves culturing the target cell with the above described radiolabeled immunotoxin or radiolabeled multimeric immunotoxin.

Another embodiment of the invention is a method that includes: (a) identifying a subject suspected of having a pathogenic cell disease; and (b) administering to the subject a radiolabeled immunotoxin that contains a toxic domain, a targeting domain, and at least one radionuclide atom. The targeting domain can be a sFv antibody fragment that binds to a target molecule on a target cell in the subject. The toxic domain can be any of the toxic polypeptides listed above, the target cell can be any of those listed above, and the target molecule can be any of those listed above. The method can be a method of killing a target cell in the subject. In such methods, the radionuclide could be ⁹⁰Y, ¹⁸⁶Re, ¹⁸⁸Re, ⁶⁴Cu, ⁶⁷Cu, ²¹²Pb, ²¹²Bi, ²¹³Bi, ¹²³I, ¹²⁵I, ¹³¹I, ²¹¹At, ³²P, ¹⁷⁷Lu, ⁴⁷Sc, ¹⁰⁵Rh, ¹⁰⁹Pd, ¹⁵³Sm, or ¹⁹⁹Au. Alternatively, the method can be an imaging method and, in this case, the radionuclide can be, for example, ¹⁸⁶Re, ¹⁸⁸Re, ⁶⁴Cu, ⁶⁷Cu, ²¹²Bi, ¹²³I, ¹³¹I, ²¹¹At, ¹⁷⁷Lu, ⁴⁷Sc, ¹⁰⁵Rh, ¹⁰⁹Pd, ¹⁵³Sm, ¹⁹⁹Au, ^{99m}Tc, ¹¹¹In, ¹²⁴I, ¹⁸F, ¹¹C, ¹⁹⁸Au, ⁷⁵Br, ⁷⁶Br, ⁷⁷Br, ¹³N, ^{34m}Cl, ³⁸Cl, ^{52m}Mn, ⁵⁵Co, ⁶²Cu, ⁶⁸Ga, ⁷²As, ⁷⁶As, ⁷²Se, ⁷³Se, or ⁷⁵Se.

The invention also embraces methods of making a radiolabeled immunotoxin. Such a method can involve, for example, the steps of:(a) providing a cell containing a vector that contains a nucleic acid sequence encoding a protein, with the nucleic acid sequence being operably linked to a transcriptional regulatory element (TRE);(b) culturing the cell;(c) extracting the protein from the culture; and (d) attaching at least one radionuclide atom to the protein. The protein can contain a toxic domain and a targeting domain and the targeting domain can be a sFv antibody fragment that binds to a target molecule on a target cell, with the target molecule preferably not being a polypeptide of the T cell CD3 complex. In a more preferred embodiment the target molecule is not the ε chain of the T cell CD3 complex. Alternatively, the method of making the radiolabeled immunotoxin can involve: (a) providing a protein that contains a toxic domain and a targeting domain; and (b) attaching at least one radionuclide atom to the protein. The targeting domain can be a sFv antibody fragment that binds to a target molecule on a target cell, with the target molecule preferably not being a polypeptide of the T cell CD3 complex. In a more preferred embodiment the target molecule is not the ε chain of the T cell CD3 complex.

The invention also features a method of making a radiolabeled multimeric immunotoxin. The method can involve, for example, the steps of: (a) providing one or more cells, each of the cells containing a nucleic acid sequence encoding a monomer with a different amino acid sequence, with the nucleic acid sequence being operably linked to a TRE; (b) separately culturing each of the one or more cells; (c) extracting the monomer from each of the cultures; (d) exposing the monomers to conditions which allow multimerization of the monomers to form a multimer comprising at least two monomers; and (e) attaching at least one radionuclide atom to the multimer. Each monomer can contain a targeting domain and a toxic domain and the targeting domain can bind to a target molecule on a target cell. It is understood that step (d) includes mixing different monomers. Alternatively, the method of making a multimeric radiolabeled immunotoxin can involve: (a) providing a multimeric protein; and (b) attaching at least one radionuclide atom to the multimeric protein. The multimeric protein contains at least two monomers, each monomer can contain a targeting domain and a toxic domain and can be physically associated with the other monomers, and the targeting domain can bind to a target molecule on a target cell.

“Polypeptide” and “protein” are used interchangeably and mean any peptide-linked chain of amino acids, regardless of length or post-translational modification.

As used herein, “operably linked” means incorporated into a genetic construct so that expression control sequences effectively control expression of a coding sequence of interest.

As used herein, the term “antibody fragments” refers to antigen-binding fragments, e.g., Fab, F(ab′)₂, Fv, and single-chain Fv (sFv) fragments. Also included are chimeric antibody fragments in which the regions involved in antigen binding (e.g., complementarity determining regions (CDR) 1, 2, and 3) are from an antibody produced in a first species (e.g., a mouse or a hamster) and the regions not involved in antigen binding (e.g., framework regions) are from an antibody produced in a second species (e.g., a human).

As used herein, a “functional fragment” of a toxic polypeptide for use as a toxic domain in the RIT and RMIT of the invention is a fragment of the toxic polypeptide shorter than the full-length, wild-type toxic polypeptide but which has at least 5% (e.g., 10%, 20%, 30%, 40%, 50%, 60%, 70%, 80%, 90%, 95%, 99%, 100%, or even more) of the toxic activity of the full-length, wild-type toxic polypeptide. In vitro and in vivo methods for comparing the relative toxic activity of two or more test compounds are known in the art.

As used herein, a “functional fragment” of a targeting polypeptide for use as a targeting domain in the RIT and RMIT of the invention is a fragment of the targeting polypeptide shorter than the full-length, wild-type targeting polypeptide but which has at least 5% (e.g., 10%, 20%, 30%, 40%, 50%, 60%, 70%, 80%, 90%, 95%, 99%, 100%, or even more) of the ability of the full-length, wild-type targeting polypeptide to bind to its relevant target molecule. Methods of comparing the relative ability of two or more test compounds to bind to a target molecule are well-known to artisans in the field, e.g., direct or competitive ELISA.

As used herein, a “coupling moiety” in a polypeptide is a residue that can be, but is not necessarily, an amino acid (e.g., cysteine or lysine), and which is inserted either internally or at a terminus (C or N) of the polypeptide. Coupling moieties can be residues that are present in native polypeptides (or functional fragments thereof) used as targeting or toxic domains or they can be heterologous. Coupling moieties serve as sites for joining of one polypeptide to another.

As used herein, a “heterologous moiety” in a polypeptide is a moiety that does not occur in the wild-type form(s) of the polypeptide or functional fragment(s) thereof.

As used herein, “physically associated” monomers are monomers that are either: (a) directly joined to each other by, for example, a covalent bond or interactions such as hydrophobic interactions or ionic interactions; or (b) are indirectly linked to each other by one or more intervening fusion proteins, each linked in a sequential fashion by the above bond or interactions.

As used herein, a “a pathogenic cell disease” of a subject is a disease in which the symptoms are caused, directly or indirectly, by cells in the subject acting in a fashion detrimental to the subject. Pathogenic cells can be, for example, cancer cells, benign hyperproliferative cells, autoreactive lymphoid (T and/or B) cells mediating autoimmune diseases, graft (allo- or xeno-) rejecting lymphoid cells, lymphoid cells (allogeneic or xenogeneic) mediating graft-verus-host disease (GVHD), or cells infected with microorganisms (e.g., bacteria, fungi, yeast, viruses, mycoplasma, or protozoa).

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., killing cancer cells in mammalian subjects, will be apparent from the following description, from the drawings and from the claims.

### BRIEF DESCRIPTION OF THE DRAWINGS

FIG. 1 is a line graph showing the in vitro cytotoxic effect of unlabeled IT DTe23, ¹²⁵I, labeled DTe23, and ^{99m}Tc labeled DTe23 on BT-474 human breast cancer cells.

FIG. 2 is a line graph showing the in vitro cytotoxic effect of unlabeled IT DTe23, ¹²⁵I labeled DTe23, and ^{99m}Tc labeled DTe23 on SKOV3.ip1 human ovarian cancer cells.

FIG. 3 is a line graph showing the in vitro cytotoxic effect of unlabeled IT DTe23, ¹²⁵I, labeled DTe23, and ^{99m}Tc labeled DTe23 on LS174T human colon cancer cells.

FIGS. 4A and 4B are A₂₈₀ and radioactivity traces from sequential preparative high pressure liquid chromatography (HPLC) separations starting with the radiolabeling reaction mixture in which the IT (DTe23) protein was labeled with ¹⁸⁸Re.

FIG. 5 is a line graph showing the in vitro cytotoxic effect on BT-474 human breast cancer cells of semi-purified ¹⁸⁸Re labeled DTe23 (“crude prep”) and a fraction (“fraction A”) containing purified ¹⁸⁸Re labeled DTe23.

FIG. 6 is a line graph showing the in vitro cytotoxic effect on LS174T human colon cancer cells of semi-purified ¹⁸⁸Re labeled DTe23 (“crude prep”) and a fraction (“fraction A”) containing purified ¹⁸⁸Re labeled DTe23.

FIG. 7 is a line graph showing the in vitro cytotoxic effect on SKOV3.ip1 human ovarian cancer cells of semi-purified ¹⁸⁸Re labeled DTe23 (“crude prep”) and a fraction (“fraction A”) containing purified ¹⁸⁸Re labeled DTe23.

FIG. 8 is a bar graph showing the distribution (expressed as a percent of the injected dose per gram of tissue (“%ID/g”)) after intraperitoneal injection of semi-purified ¹⁸⁸Re labeled DTe23 (“crude unpurified”; i.e., the CRUDE peak from FIG. 4A) or more purified ¹⁸⁸Re labeled DTe23 (“purified”; i.e., peak A from FIG. 4B) in various tissues of nude mice bearing an intraperitoneal tumor of LS147T human colon cancer cells (tissues: BL, blood: LU, lung; LI, liver; SI, small intestine; SP, spleen; KI, kidney; TU, tumor).

FIG. 9 is a line graph showing the survival of two groups of athymic nude mice (n=5 mice per group) bearing SKOV3.ip1 human ovarian cancer xenografts in the peritoneum and injected intraperitoneally (i.p.) with either unlabeled DTe23 (“DTe23”; 57 μg and 20 μg on days 7 and 9, respectively, after tumor cell injection) or ¹³¹I labeled DTe23 (“131I-DTe23”; 450 μCi (57 μg) and 250 μCi (20 μg) on days 7 and 9, respectively, after tumor cell injection).

FIG. 10 is a line graph showing the in vitro cytotoxic effect on BT-474 human breast cancer cells of ⁶⁴Cu-trisuccin-DT₃₉₀e23) (“64Cu-DTe23”) or unlabeled DTe23 (“DTe23”).

FIG. 11 is a line graph showing the survival of two groups of athymic nude mice bearing LS174T human colon cancer xenografts in the peritoneum and injected i.p. on day 4 with either 19.5 μg unlabeled DTe23 (“DTe23”) or 200 μCi (19.5 μg) ⁶⁴Cu-trisuccin-DTe23 (“Cu-64-DTe23”).

### DESCRIPTION OF THE PREFERRED EMBODIMENTS

The invention is based upon experiments with a RIT labeled with three different radionuclides. The RIT contained: (a) a toxic domain (a portion of diphtheria toxin (DT)); (b) a targeting domain which was a single-chain Fv fragment (sFv) derived from antibody specific for erbB2 (Her-2/neu), a protein expressed on the surface of several cancer cell types (e.g. breast, colon and ovarian cancer cells); and (c) radionuclide (¹²⁵I, ^{99m}Tc, or ¹⁸⁸Re) atoms. The RIT retained substantially all of the cytotoxic activity of the unlabeled parent molecules and, in some cases, showed significantly greater activity. Furthermore, in vivo biodistribution studies showed localization of a RIT to a tumor. In addition, in vivo therapy studies showed enhanced therapeutic efficacy of two different RIT compared to unlabeled IT against two different tumors.

While the invention is not limited by any particular mechanism of action, prior studies indicate that the toxic domains used in the RIT of the invention kill target cells by inhibiting protein synthesis and the radiation emitted by the radiolabels kill them by causing, directly or indirectly, DNA damage. RIT have the advantage over unlabeled IT of being able to kill cells to which the RIT is not bound but which are sufficiently close to a cell to which the RIT is bound to be affected by the radiation emitted by the radionuclide. RIT also have advantages conveyed by combining the cytotoxic activity of a radionuclide and that of a toxin in the same molecule. The use of a RIT for killing a target cell of interest avoids the competition for an appropriate cellular ligand on a target cell that would occur between a first IT containing a targeting domain of interest and a radiolabel and second IT containing the same targeting domain and a toxin when the target cell is exposed to the two individual IT. In addition, combining all three components into a single molecule or molecular complex is both more logistically efficient and more cost efficient than producing, packaging, and, for example, administering to appropriate subjects (e.g., cancer patients) two separate molecular entities.

A. RIT

The RIT of the invention contain a targeting domain linked to a toxic domain and at least one radionuclide atom (see below). The RIT can also contain one or more (e.g., one, two, three, four, five, six, seven, eight, nine, or ten) additional targeting domains or one or more (e.g., one, two, three, four, five, six, seven, eight, nine, or ten) additional toxic domains. Targeting domains, toxic domains, and radionuclides are described in the following subsections.

A.1 Targeting domains

A targeting domain for use in the RIT of the invention can be any polypeptide (or a functional fragment thereof) that has significant binding affinity for a target molecule on the surface of a target cell (e.g., a tumor cell or an infected cell). Thus, for example, where the molecule on the surface of the target cells is a receptor, the targeting domain will be a ligand for the receptor, and where the molecule on the surface of the target cells is a ligand, the targeting domain will be a receptor for the ligand. Targeting domains can also be functional fragments of appropriate polypeptides (see below).

The invention thus includes as targeting domains antibody fragments specific for an antigen on the surface of a target cell. Antibody fragments used as targeting domains in the RIT of the invention contain the antigen combining site of an antibody molecule. The antibody fragments do not generally contain the whole constant region of either the heavy (H) or light (L) chain of an antibody molecule. However the antibody fragments can contain segments of the constant region of either or both the H and L chain. These constant region segments can be from the N-terminal end of the constant region or from any other part of the constant regions, e.g., the hinge region of IgG or IgA heavy chains. They can also optionally contain one or more (e.g., two, three, four, five, six, seven, eight, nine, ten, 11, 12, 13, 14, 15, 17, 20, 25, 30, 40, or more) constant (C) region amino acids.

An antibody fragment for use as a targeting domain contains V regions of both H and L chains of an antibody molecule. In addition, it can contain: (a) all or some of the J regions of both or either of the H and the L chain; and (b) the D region of the H chain. In general, the antibody will contain the CDR3 amino acid residues of an antibody molecule, i.e., those amino acids encoded by nucleotides at the C-termini of the V region gene segments, and/or P or N nucleotides inserted at the junctions of either the V and J, the V and D, or the D and J region gene segments during somatic B cell gene rearrangements necessary for the generation of functional genes encoding H and L chains. The antibody fragments can contain more than one (e.g., 2, 3, 4, or 5) antigen combining site, i.e., the above-described units containing components from both a H chain and a L chain.

Preferred antibody fragments are sFv fragments containing the V and, optimally, the CDR3 regions, of H and L chains joined by a flexible linker peptide. The term V region, as used in all subsequent text, unless otherwise stated, will be understood to include V regions alone and V regions and P/N nucleotides, and/or D regions, and/or J regions. They can also optionally contain one or more (e.g., two, three, four, five, six, seven, eight, nine, ten, 11, 12, 13, 14, 15, 17, 20, 25, 30, 40, or more) C region amino acids. Generally, but not necessarily, the heavy chain variable region (VH) will be C-terminal of the light chain variable region (VL). Linker peptides joining VH and VL regions can be 1 to about 30, even 50, amino acids long and can contain any amino acids. In general, a relatively large proportion (e.g., 20%, 40%, 60%, 80%, 90%, or 100%) of the amino acid residues in the linker will be glycine and/or serine residues. Such linkers can contain, for example, one or more (e.g., two, three, four, five, six, seven, eight, nine, ten, or more) gly-gly-gly-ser (GGGS) units.

Antibody fragments can be specific for (i.e., will have significant binding affinity for) a molecule expressed on the surface of a target cell of interest. The target molecules can be any type of protein but can also be carbohydrates (free or bound to proteins in the form of glycoproteins) or lipids (free or bound to proteins in the form of lipoproteins), e.g., gangliosides. Thus, targeting domain antibody fragments can have specific binding affinity for molecules such as T cell surface molecules (e.g., CD3 polypeptides, CD4, CD8, CD2, CD5, CD7, T cell receptor (TCR) α-chain, or TCR β-chain), B cell surface molecules (e.g., CD5, CD19, CD20, CD22, Ig molecules), other hematopoieteic cell surface molecules such as CD33, CD37, or CD45, cytokine or growth factor receptors (e.g., polypeptides of receptors for interleukin-(IL-)2 (e.g., CD25), IL-3, IL-13, IL-4, vascular endothelial growth factor (VEGF; e.g., VEGF, VEGF A, VEGF B, VEGF C, or VEGF D), granulocyte macrophage-colony stimulating factor (GM-CSF), or epidermal growth factor (EGF), molecules expressed on tumor cells (e.g., any of the molecules listed above as well as others known in the art, e.g., melanoma, breast, ovarian, or colon cancer antigens), and molecules expressed on the surface of infected target cells (e.g., viral proteins and glycoproteins). Target molecules on tumor cells are preferably expressed at a level and/or density at least two fold (e.g., three-fold, four-fold, five-fold, seven-fold, ten-fold, 20-fold, 30-fold, 50-fold, 80-fold, 100-fold, 200-fold, 500-fold, 1,000-fold, or even 10,000-fold) higher than on their normal cell counterparts. Tumor target molecules of interest include, in addition to the above-listed lymphoid cell (T and B) molecules, hematopoetic cell molecules, and cytokine or growth factor receptor molecules, mucin molecules (e.g., MUC-1, MUC-2, or MUC-3), Her-2/neu, carcinoembryonic antigen (CEA), prostate-specific antigen (PSA),a folate binding receptor polypeptide, A33 alpha fetoprotein, CA-125 glycoprotein, colon-specific antigen p, ferritin, p-glycoprotein, G250, OA3, PEM glycoprotein, L6 antigen, 19-9, P97, placental alkaline phosphatase, 7E11-C5, 17-1A, TAG-72, 40 kDa glycoprotein, URO-8, a tyrosinase, an insulin receptor polypeptide, an insulin-like growth factor receptor polypeptide, a transferrin receptor polypeptide, an estrogen receptor polypeptide, a MAGE polypeptide (e.g., MAGE-1, MAGE-3, or MAGE-6), a BAGE polypeptide, a GAGE polypeptide (e.g. GAGE-1, GAGE-2, GAGE-3, GAGE-4, GAGE-5, or GAGE-6), a RAGE polypeptide, a PRAME polypeptide, or a GnTV polypeptide.

The targeting domains can also be immunoglobulin (Ig) molecules of irrelevant specificity (or immunoglobulin molecule fragments that include or contain only an Fc portion) that can bind to an Fc receptor (FcR) on the surface of a target cell (e.g., a tumor cell).

The targeting domains can be cytokines (e.g., IL-1, IL-2, IL-3, IL-4, IL-5, IL-6, IL-7, IL-8, IL-10, IL-12, IL-13, IL-15, the interferons (α, β, and γ), TNF-α, a VEGF (e.g., VEGF, VEGF A, VEGF B, VEGF C, or VEGF D), EGF, colony stimulating factors (e.g., GM-CSF), hormones (e.g., insulin, estrogen, or growth hormone), ligands for signal transduction receptors (e.g., CD40 ligand, an MHC class I molecule or fragments of an MHC molecule involved in binding to CD8, an MHC class II molecule or the fragment of an MHC class II molecule involved in binding to CD4), or ligands for adhesion receptors, e.g., ICAM-1, ICAM-2, or fibronectin or a domain (e.g., one containing one or more of the “Arg-Gly-Asp” repeats) of fibronectin involved in binding to integrin molecules. In addition a targeting domain could be Fas or Fas ligand or other death domain containing polypeptides (e.g., members of the TNF receptor family) or ligands for such polypeptides (e.g., TNF-α, or TWEAK)

Furthermore, in certain B cell lymphomas, the specificity of the cell surface Ig molecules has been defined. Thus, where such B cell lymphoma cells are the target cells, a RIT of the invention could include, as a targeting domain, the antigen or a fragment containing the relevant antigenic determinant for which the surface Ig on the lymphoma cells is specific and thus has significant binding affinity. Such a strategy can also be used to kill B cells which are involved in the pathology of an autoimmune disease (e.g., systemic lupus erythematosus (SLE) or myasthenia gravis (MG)) and which express on their surface an Ig receptor specific for an autoantigen.

Similarly, malignant T cells or autoreactive T cells expressing a TCR of known specificity can be killed with an immunotoxin protein containing, as the targeting domain, a soluble MHC (class I or class II) molecule, an active (i.e., TCR-binding) fragment of such a molecule, or a multimer (e.g., a dimer, trimer, tetramer, pentamer, or hexamer) of either the MHC molecule or the active fragment. All these MHC or MHC-derived molecules can contain, within their antigenic peptide-binding clefts, an appropriate antigenic peptide. Appropriate peptide fragments could be from collagen (in the case of RA), insulin (in IDDM), or myelin basic protein (in MS). Tetramers of MHC class I molecules containing an HIV-1-derived or an influenza virus-derived peptide have been shown to bind to CD8+ T cells of the appropriate specificity [Altman et al. (1996), Science 274:94-96; Ogg et al. (1998), Science 279:2103-2106], and corresponding MHC class II multimers would be expected to be similarly useful with CD4+ T cells. Such complexes could be produced by chemical cross-linking of purified MHC class II molecules assembled in the presence of a peptide of interest or by modification of already established recombinant techniques for the production of MHC class II molecules containing a single defined peptide [Kazono et al. (1994), Nature 369:151-154; Gauthier et al. (1998), Proc. Natl. Acad. Sci. U.S.A. 95:11828-11833]. The MHC class II molecule monomers of such multimers can be native molecules composed of full-length α and β chains. Alternatively, they can be molecules containing either the extracellular domains of the α and β chains or the α and β chain domains that form the “walls” and “floor” of the peptide-binding cleft.

In addition, the targeting domain could be a polypeptide or functional fragment that binds to a molecule produced by or whose expression is induced by a microorganism infecting a target cell. Thus, for example, where the target cell is infected by HIV, the targeting domain could be an HIV envelope glycoprotein binding molecule such as CD4, CCR4, CCR5, or a functional fragment of any of these.

The invention also includes artificial targeting domains. Thus, for example, a targeting domain can contain one or more different polypeptides, or functional fragments thereof, that bind to a target cell of interest. Thus, for example, a given targeting domain could contain whole or subregions of both IL-2 and IL-4 molecules or both CD4 and CCR4 molecules. The subregions selected would be those involved in binding to the target cell of interest. Methods of identifying such “binding” subregions are known in the art. In addition, a particular binding domain can contain one or more (e.g., 2,3, 4, 6, 8, 10, 15, or 20) repeats of one or more (e.g., 2, 3, 4, 6, 8, 15, or 20) binding subregions of one or more (e.g., 2, 3, 4, or 6) polypeptides that bind to a target cell of interest.

The targeting domains can be molecules (e.g., sFv) of any species, e.g., a human, non-human primate (e.g., monkey), mouse, rat, guinea pig, hamster, cow, sheep, goat, horse, pig, rabbit, dog, or cat.

The amino acid sequence of the targeting domains of the invention can be identical to the wild-type sequence of appropriate polypeptide. Alternatively, the targeting domain can contain deletions, additions, or substitutions. All that is required is that the targeting domain have at least 5% (e.g., 10%, 20%, 30%, 40%, 50%, 60%, 70%, 80%, 90%, 95%, 99%, 100%, or even more) of the ability of the wild-type polypeptide to bind to the target molecule. Methods of comparing the relative ability of two or more molecules to bind to cells are known in the art. Substitutions will preferably be conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

Particularly useful as polypeptide targeting domains are those whose nucleotide sequences have been defined and made public. Indeed, the nucleotide sequences encoding the H and L chains of many appropriate antibodies have been defined and are available to the public in, for example, scientific publications or data bases accessible to the public by mail or the internet. For example, the nucleic acid sequences (and references disclosing them) encoding the following polypeptides were obtained from GenBank at the National Center for Biotechnology Information, National Library of Medicine, Bethesda, Md.: VH and VL of an antibody specific for human CD4 [Weissenhorn et al. (1992) Gene 121(2):271-278]; VH and VL of an antibody specific for human CD3 [GenBank Accession Nos. AF078547 and AF078546]; VH and VL of an antibody specific for human CD7 [Heinrich et al. (1989) J. Immunol. 143:3589-3597]; VH and VL of an antibody specific for MUC-1 [Denton et al. (1995) Eur. J. Cancer 31A:214-221]; VH and VL of an antibody specific for CEA [Cabilly et al. (1984) Proc. Natl. Acad. Sci. U.S.A, 81:3273-3277]; human IL-1α [Gubler et al. (1986) J. Immunol. 136(7):2492-2497]; human IL-3 [Yang et al. (1986) Cell 47(1):3-10]; human IL-4 (genomic DNA sequence) [Arai et al. (1989) J. Immunol. 142(1):274-282]; human IL-4 (cDNA sequence) [Yokota et al. (1986) Proc. Natl. Acad. Sci. U.S.A. 83(16):5894-5898]; human GM-CSF [Wong et al. (1985) Science 228(4701):81-815]; human VEGF [Weindel et al. (1992) Biochem. Biophys. Res. Comm. 183(3):1167-1174];

human EGF [Bell et al. (1986) Nucleic Acids Res. 14(21):8427-8446]; and human CD40 ligand [Graf et al. 5(1992) Eur. J. Immunol. 22(12):3191-3194].

However, the invention is not limited to the use of targeting domains whose nucleotide sequences are currently available. Methods of cloning nucleic acid molecules encoding polypeptides and establishing their nucleotide sequences are known in the art [e.g., Maniatis et al., Molecular Cloning: A Laboratory Manual (Cold Spring Harbor Laboratory, N.Y., 1989) and Ausubel et al. Current Protocols in Molecular Biology (Green Publishing Associates and Wiley Interscience, N.Y., 1989)].

A.2 Toxic domains

Toxic domains useful in the invention can be any toxic polypeptide that mediates a cytotoxic effect on a cell. Preferred toxic polypeptides include ribosome inactivating proteins, e.g., plant toxins such as an A chain toxin (e.g., ricin A chain), saporin, bryodin, gelonin, abrin, or pokeweed antiviral protein (PAP), fungal toxins such as α-sarcin, aspergillin, or restrictocin, bacterial toxins such as DT or Pseudomonas exotoxin A, or a ribonuclease such as placental ribonuclease or angiogenin. Other useful toxic polypeptides are the pro-apoptotic polypeptides, e.g., Bax, Bad, Bak, Bim, Bik, Bok, or Hrk. As with the targeting domains, the invention includes the use of functional fragments of any of the polypeptides. Furthermore, a particular toxic domain can include one or more (e.g., 2, 3, 4, or 6) of the toxins or functional fragments of the toxins. In addition, more than one functional fragment (e.g. 2, 3, 4, 6, 8, 10, 15, or 20) of one or more (e.g., 2, 3, 4, or 6) toxins can be included in the toxic domain. Where repeats are included, they can be immediately adjacent to each other, separated by one or more targeting fragments, or separated by a linker peptide as described above.

The amino acid sequence of the toxic domains of the invention can be identical to the wild-type sequence of appropriate polypeptide. Alternatively, the toxic domain can contain deletions, additions, or substitutions. All that is required is that the toxic domain have at least 5% (e.g., 10%, 20%, 30%, 40%, 50%, 60%, 70%, 80%, 90%, 95%, 99%, 100%, or even more) of the ability of the wild-type polypeptide to kill relevant target cells. It could be desirable, for example, to delete a region in a toxic polypeptide that mediates non-specific binding to cell surfaces. Substitutions will preferably be conservative substitutions (see above).

Particularly useful as toxic domains are those toxic polypeptides whose nucleotide sequences have been defined and made public. Indeed, the nucleotide sequences encoding many of the toxic polypeptides listed above have been defined and are available to the public. For example, the nucleic acid sequences (and references disclosing them) encoding the following toxic polypeptides were obtained from GenBank at the National Center for Biotechnology Information, National Library of Medicine, Bethesda, Md.: gelonin [Nolan et al. (1993) Gene 134(2):223-227]; saporin [Fordham-Skelton et al. (1991) Mol. Gen. Genet. 229(3);460-466]; ricin A-chain [Shire et al. (1990) Gene 93:183-188]; α-sarcin [Oka et al. (1990) Nucleic Acids Res. 18(7):1897; restrictocin [Lamy et al. (1991) Mol. Microbiol. 5(7):1811-1815]; and angiogenin [Kurachi et al. (1985) Biochemistry 24(20):5494-5499].

However, the invention is not limited to the use of toxic domains whose nucleotide sequences are currently available. Methods of cloning nucleic sequences encoding known polypeptides and establishing their nucleotide sequences are known in the art [Maniatis et al., supra, Ausubel et al., supra].

Toxic and targeting domains can be disposed in any convenient orientation with respect to each other in the RIT of the invention. Thus, the toxic domain can be N-terminal of the targeting domain or vice versa. The two domains can be immediately adjacent to each or they can be separated by a linker (see above).

Smaller IT proteins (less than 100 amino acids long) can be conveniently synthesized by standard chemical means. In addition, IT polypeptides can be produced by standard in vitro recombinant DNA techniques and in vivo recombination/genetic recombination (e.g., transgenesis), using the nucleotide sequences encoding the appropriate polypeptides or peptides. The IT fusion proteins can also be made by a combination of chemical and recombinant methods.

Methods well known to those skilled in the art can be used to construct expression vectors containing relevant coding sequences and appropriate transcriptional/translational control signals. See, for example, the techniques described in Sambrook et al., Molecular Cloning: A Laboratory Manual (2nd Ed.) [Cold Spring Harbor Laboratory, N.Y., 1989], and Ausubel et al., Current Protocols in Molecular Biology, [Green Publishing Associates and Wiley Interscience, N.Y., 1989].

Expression systems that may be used for small or large scale production of the IT proteins include, but are not limited to, microorganisms such as bacteria (for example, E. coli and B. subtilis) transformed with recombinant bacteriophage DNA, plasmid DNA, or cosmid DNA expression vectors containing the nucleic acid molecules of the invention; yeast (for example, Saccharomyces and Pichia) transformed with recombinant yeast expression vectors containing the nucleic acid molecules of the invention (see below); insect cell systems infected with recombinant virus expression vectors (for example, baculovirus) containing the nucleic acid molecules of the invention; plant cell systems infected with recombinant virus expression vectors (for example, cauliflower mosaic virus (CaMV) and tobacco mosaic virus (TMV)) or transformed with recombinant plasmid expression vectors (for example, Ti plasmid) containing fusion protein nucleotide sequences; or mammalian cell systems (for example, COS, CHO, BHK, 293, VERO, HeLa, MDCK, WI38, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter). Also useful as host cells are primary or secondary cells obtained directly from a mammal, transfected with a plasmid vector or infected with a viral vector.

RIT of the invention also include those described above, but which contain additional amino acid segments. Thus the RIT can contain, for example, a hydrophobic signal peptide. The signal peptide is generally immediately N-terminal of the mature polypeptide (fusion protein) but can be separated from it by one or more (e.g., 2, 3, 4, 6, 8, 10, 15 or 20) amino acids, provided that the leader sequence is in frame with the nucleic acid sequence encoding the fusion protein. The signal peptide, which is generally cleaved from proteins prior to secretion, directs proteins into the lumen of an appropriate cell's endoplasmic reticulum (ER) during translation and the proteins are then secreted, via secretory vesicles, into the environment of the cell. In this way, the cells producing IT proteins remain viable since interaction of the toxic domain of IT protein with the protein synthetic machinery in the cytosol of the cell is prevented by the membrane bilayers of the ER and secretory vesicles. Useful leader peptides can be the native leader peptide of the relevant targeting domain (e.g., VH or VL) or a functional fragment of the native leader. Alternatively, the leader can be that of another exported polypeptide. For example, the signal peptide can have the amino acid sequence MAISGVPVLGFFIIAVLMSAQESWA (SEQ ID NO: 1). In addition, the peptide sequence KDEL (SEQ ID NO: 2) has been shown to act as a retention signal for the ER.

The RIT of the invention can also be modified for in vivo use by the addition, at the amino- and/or carboxyl-terminal ends, of a blocking agent to facilitate survival of the relevant polypeptide in vivo. This can be useful in those situations in which the polypeptide termini tend to be degraded by proteases prior to cellular uptake. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to artisans of average skill.

Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety.

A.3 Radionuclides

Radionuclides useful for labeling proteins to be used for therapeutic and/or imaging purposes are known in the art (see, for example, U.S. Pat. No. 6,001,329 which is incorporated herein by reference in its entirety). Each IT molecule of the invention includes at least one (e.g., one, two, three, four, five, six, seven, eight, nine, ten, 20, 30, 40, 50, 100, 200, or more) radionuclide atoms. Methods of varying and determining the average number of radionuclide atoms bound to a polypeptide of interest are known in the art. From a knowledge of the weight of protein present in the sample, the molecular weight of the protein, the half-life of the radionuclide, and the radioactivity of the sample, it is possible to calculate the average number of radionuclide atoms bound per protein molecule in the sample. This would initially involve calculating the number of radionuclide atoms in the sample with the following two formulae:

N=D/λ

λ=0.693/t_{½}

where N is the number of atoms in the sample;

D is the disintegration rate for the radionuclide (derivable from the radioactivity of the sample);

λ is the decay constant for the radionuclide; and

t_{½} is the half-life of the radionuclide.

The radionuclide atoms can be bound by covalent or non-covalent (e.g., ionic or hydrophobic bonds) to the IT polypeptide. They can be bound to any part of the IT polypeptide, e.g., the targeting domain or the toxic domain. All that is required is that the radiolabeled targeting domain or toxic domain have at least 5% (e.g., 10%, 20%, 30%, 40%, 50%, 60%, 70%, 80%, 90%, 95%, 100%, or more) of the activity of the corresponding unlabeled targeting domain or toxic domain, respectively. The radionuclide atom can be directly bound to the protein backbone of the IT, e.g., in some applications of ¹²³I, ¹²⁵I, or ¹³¹I. Alternatively, the radionuclide atom can be part of a larger molecule ( e.g., ¹²⁵I in meta-[¹²⁵I]iodophenyl-N-hydroxysuccinimide ([¹²⁵I]mIPNHS) which binds via free amino groups to form meta-iodophenyl (mIP) derivatives of relevant proteins [Rogers et al. (1997) J. Nucl. Med. 38:1221-1229]) or chelate (e.g., radioactive metal atoms such as ^{99m}Tc, ¹⁸⁸Re, ¹⁸⁶Re, ⁹⁰Y, ²¹²Pb ²¹²Bi ⁶⁴Cu, ⁶⁷Cu, ¹⁷⁷Lu, ⁴⁷Sc, ¹⁰⁵Rh, ¹⁰⁹Pd, ¹⁵³Sm, ¹⁹⁹Au chelated to, for example, Hhydroxamic acids, DOTA, or DTPA) which is in turn bound to the protein backbone. However, radioactive metal atoms can also bind directly to the protein via, for example, free sulfhydryl groups on the protein. ³²P can be attached to the RIT, for example, in the form of phosphate groups using amino acid residues in the IT polypeptide such as serine, threonine, or tyrosine. Methods of attaching the radionuclide atoms or larger molecules/chelates containing them to the IT protein backbones are known in the art. Such methods involve incubating the IT protein with the radionuclide under conditions (e.g., pH, salt concentration, and/or temperature) which facilitate binding of the radionuclide atom or radionuclide atom-containing molecule or chelate to the IT protein (see, e.g. U.S. Pat. No. 6,001,329). Where the RIT contains more than one radionuclide atom, the various radionuclide atoms can be either all the same radionuclide, all different radionuclides, or some the same and some different radionuclides. The radionuclides can emit α-, β-, or γ- radiation or a combination of two or more of these types of irradiation.

B. Radiolabeled Multimeric IT

The radiolabeled multimeric IT (RMIT) of the invention will contain two or more (e.g., three, four, five, six, or eight) of the IT polypeptides (“monomers”) described above and one or more (e.g., one, two, three, four, five, six, seven, eight, nine, or ten) radionuclide atoms. Multimeric IT without attached radionuclide atoms and experiments (in vitro and in vivo) using them are described in detail in co-pending U.S. application no. 09/440,344 which is incorporated herein by reference in its entirety. In a preferred embodiment, the RMIT are dimeric, i.e., they contain two IT monomers.

Each monomer of the RMIT can be identical, i.e., contain the same targeting and toxic domains and have the same amino acid sequence. Alternatively, they can be different. Thus, they can contain, for example, the same targeting domains but different toxic domains, different targeting domains but the same toxic domains, or different targeting domains and different toxic domains. Where different targeting domains are used, they will generally have significant binding affinity for either the same cell-surface molecule or for different molecules on the surface of the same cell.

The monomers can be linked to each other by methods known in the art. For example, a terminal or internal cysteine residue on one monomer can be utilized to form a disulfide bond with a terminal or internal cysteine residue on another monomer.

Monomers can also be cross-linked using any of a number of known chemical cross linkers. Examples of such reagents are those which link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4succinimidyloxycarbonyl-α-methyl-α(2-pyridyldithio)toluene (SMPT), forms such a linkage between two monomers utilizing a terminal lysine on one of the monomers and a terminal cysteine on the other. Heterobifunctional reagents which cross-link by a different coupling moiety on each monomer polypeptide can be particularly useful in generating, for example, dimeric RMIT involving two different monomers. Thus, the coupling moiety on one monomer could be a cysteine residue and on the other a lysine residue. In this way, the resulting dimers will be heterodimers rather than either homodimers or a mixture of homodimers and heterodimers. Other useful cross-linkers, which are listed in the Pierce Products catalog (1999/2000), include, without limitation, reagents which link two amino groups (e.g., N-5-Azido-2-nitrobenzoyloxysuccinimide), two sulfhydryl groups (e.g., 1,4-Bis-maleimidobutane) an amino group and a sulfhydryl group (e.g., m-Maleimidobenzoyl-N-hydroxysuccinimide ester), an amino group and a carboxyl group (e.g., 4-[p-Azidosalicylamido]butylamine), and an amino group and a guanadium group that is present in the side chain of arginine (e.g., p-Azidophenyl glyoxal monohydrate).

While these cross-linking methods can involve residues (“coupling moieties”) that are native to either of the domains of the monomers, they can also be used to cross-link non-native (“heterologous”) residues incorporated into the polypeptide chains. While not necessarily the case, such residues will generally be amino acids (e.g., cysteine, lysine, arginine, or any N-terminal amino acid). Non-amino acid moieties include, without limitation, carbohydrates (e.g., on glycoproteins) in which, for example, vicinal diols are employed [Chamow et al. (1992) J. Biol. Chem. 267, 15916-159223. The cross-linking agent 4-(4-N-maleimidophenyl)butyric acid hydrazide (MPBH), for example, can be used to cross-link a carbohydrate residue on one monomer and a sulfhydryl group on another. They can be added during, for example, chemical synthesis of a monomer or a part of the monomer. Alternatively, they can be added by standard recombinant nucleic acid techniques known in the art.

The heterologous coupling moieties can be positioned anywhere in the monomer fusion proteins, provided that the activity of the resulting RMIT is not compromised. Thus, the linkage must not result in disruption of the structure of a targeting domain such that it is substantially unable to bind to the cell-surface molecule for which it is specific. Furthermore, the linkage must not result in the disruption of the structure of the toxic domain such that it is substantially unable to kill its respective target cell. Using standard binding and toxicity assays known to those in the art, candidate RMIT employing linkages involving different residues on the monomers can be tested for their ability to bind and kill target cells of interest. Using molecular modeling techniques, it will frequently be possible to predict regions on a targeting domain or toxic domain that would be appropriate for the insertion of moieties by which inter-monomer linkages could be formed. Thus, for example, regions predicted to be on the exterior surface of a targeting domain, but unlikely to be involved in binding to a target molecule, could be useful regions in which to an insert an appropriate moiety in the targeting domain. Similarly, regions predicted to be on exterior surface of a toxic domain, but unlikely to be involved in the toxic activity, could be useful regions in which to an insert an appropriate moiety in the toxic domain.

The coupling moieties will preferably be at the termini (C or N) of the monomers. They can be, as indicated above, a cysteine residue on each monomer, or a cysteine on one and a lysine on the other. Where they are two cysteine residues, cross-linking can be effected by, for example, exposing the monomers to oxidizing conditions.

It can be desirable in some cases to eliminate, for example, one or more native cysteine residues in a monomer in order to restrict cross-linking to only non-native moieties inserted into the monomers. A potentially troublesome cysteine could, for example, be replaced by an alanine or a tyrosine residue. This can be done by, for example, standard recombinant techniques. Naturally, these replacements should not compromise the activity of the resulting RMIT (see above).

It is understood that in RMIT containing more than two monomers, at least one of the monomers will have more than one cross-linking moiety. Such multimers can be constructed “sequentially”, such that each monomer is joined to the next such that the terminal two monomers in the chain only have one residue involved in an intermonomer bond while the “internal” monomers each have two moieties involved in inter-monomer bonds. Alternatively, one monomer could be linked to multiple (e.g., 2, 3, 4, or 5) other monomers. In these cases the first monomer would be required to contain multiple native and/or non-native cross-linkable moieties. A multimeric IT protein could also be formed by a combination of these two types of structure.

Radiolabels for the RMIT and methods of attaching them are essentially the same as those described above for RIT.

C. Methods of Killing Target Cells with RIT and RMIT

The radiolabeled immunotoxic proteins (RIT and RMIT) of the invention can be added to a cell population in vitro in order, for example, to deplete the population of cells expressing a cell surface molecule to which the targeting domain of an appropriate fusion protein binds. For example, the population of cells can be bone marrow cells from which it desired to remove T cells prior to use of the bone marrow cells for allogeneic or xenogeneic bone marrow transplantation. Alternatively, it may be desirable to deplete bone marrow cells of contaminating tumor cells prior to use of the bone marrow cells for bone marrow transplantation (autologous, allogeneic, or syngeneic) in a cancer patient. In such in vitro administrations, the cells to be depleted can be cultured with the RIT and/or RMIT to allow binding of the RIT and/or RMIT to the target cells followed by killing of the target cells.

Alternatively, a RIT or RMIT can be administered as a therapeutic agent to a subject in which it is desired to eliminate a cell population expressing a cell surface molecule to which the targeting domain of the fusion protein binds. Appropriate subjects include, without limitation, those with any of a variety of tumors (e.g., hematological cancers such as leukemias and lymphomas, neurological tumors such as astrocytomas or glioblastomas, melanoma, breast cancer, lung cancer, head and neck cancer, gastrointestinal tumors, genitourinary tumors, ovarian tumors, bone tumors, vascular tissue tumors, or any of a variety of non-malignant tumors), transplant (e.g., bone marrow, heart, kidney, liver, pancreas, or lung) recipients, those with any of a variety of autoimmune diseases (e.g., rheumatoid arthritis, insulin dependent diabetes mellitus, multiple sclerosis, myasthenia gravis, or systemic lupus erythematosus), or those with an infectious disease involving an intracellular microorganism (e.g., Mycobacterium tuberculosis, Salmonella, influenza virus, measles virus, hepatitis C virus, human immunodeficiency virus, and Plasmodium falciparum). Delivery of an appropriate RIT or RMIT to tumor cells can result in the death of a substantial number, if not all, of the tumor cells. In transplant recipients, the RIT or RMIT is delivered, for example, to T cells, thereby resulting in the death of a substantial number, if not all, of the T cells. In the case of a hematopoietic (e.g., bone marrow) cell transplant, the treatment can diminish or abrogate both host-versus-graft rejection and GVHD. In infectious diseases, the RIT or RMIT is delivered to the infected cells, thereby resulting in the death of a substantial number of, in not all, the cells and thus a substantial decrease in the number of, if not total elimination of, the microorganisms. In autoimmune diseases, the RIT or RMIT can contain, for example, a targeting domain specific for T cells (CD4+ and/or CD8+) and/or B cells capable of producing antibodies that are involved in the tissue destructive immune responses of the diseases.

In addition to their use as therapeutic agents, the RIT or RMIT of the invention can be used as imaging agents. Thus, for example, prior to administration of an therapeutic RIT or RMIT to a subject with a solid tumor, the ability of the RIT or RMIT to home to the tumor, and, if present, metastases, can be tested by administering to the subject an RIT or RMIT labeled with an appropriate imaging radionuclide (see below). The subject then undergoes an appropriate scanning procedure to measure the distribution of the imaging RIT or RMIT in the body of the subject and thereby assess the efficiency of an equivalent therapeutic RIT or RMIT to localize to the tumor and metastases if present. The invention is, however, not limited to situations in which imaging is performed preliminary to a therapeutic regimen. The imaging methods can also be performed independently of or without any subsequent therapeutic procedure(s) with the RIT or RMIT of the invention.

While the imaging methods of the invention will generally be used for subjects with tumors (malignant or non-malignant), they can also be applied in the other pathogenic cell diseases listed herein. Thus, for example, an appropriately labeled RIT or RMIT containing a targeting domain specific for a hematopoietic cell (e.g., a T cell) surface molecule (e.g., an sFv that binds to any of the hematopoietic cell surface molecules listed above or a cytokine or growth factor molecule that binds to any of the cytokine or growth factor receptors listed above) can be used to image a body region in which, for example, graft rejection, an autoimmune reaction, or an infection-related inflammatory response is occurring. Similarly, RIT or RMIT containing targeting domains that bind to cells harboring any of the infectious microorganisms listed above can be used to image body regions containing the infectious microorganisms, preferably within cells of the host subject.

Isotopes suitable for imaging purposes are not necessarily those suitable for therapeutic purposes. For use in imaging, a radionuclide must emit photons. Thus, for example, radionuclides such as ¹⁸⁶Re, ¹⁸⁸Re, ⁶⁴Cu, ⁶⁷Cu, ²¹²Bi, ¹²³I, ¹³¹I, ²¹¹At, ¹⁷⁷Lu, ⁴⁷Sc, ¹⁰⁵Rh, ¹⁰⁹Pd, ¹⁵³Sm, ¹⁹⁹Au, ^{99m}Tc, ¹¹¹In, ¹²⁴I, ¹⁸F, ¹¹C, ¹⁹⁸Au, ⁷⁵Br, ⁷⁶Br, ⁷⁷Br, ¹³N, ^{34m}Cl, ³⁸Cl, ^{52m}Mn, ⁵⁵Co, ⁶²Cu, ⁶⁸Ga, ⁷²As, ⁷⁶As, ⁷²Se, or ⁷⁵Se can be used to generate RIT or RMIT useful for the imaging methods of the invention. Methods of attaching the relevant atoms to a RIT or RMIT protein are known in the art and are similar to those described above.

Subjects receiving such treatment or being subjected to imaging can be any mammal, e.g., a human (e.g., a human cancer patient), a non-human primate (e.g., a chimpanzee, a baboon, or a rhesus monkey), a horse, a pig, a sheep, a goat, a bovine animal (e.g., a cow or a bull), a dog, a cat, a rabbit, a rat, a hamster, a guinea pig, or a mouse.

The RIT and RMIT of the invention can be provided as compositions in a pharmaceutically acceptable diluent (e.g., physiological saline). Whether provided dry or in solution, they can be prepared for storage by mixing them with any one or more of a variety of pharmaceutically acceptable carriers, excipients or stabilizers known in the art [Remington's Pharmaceutical Sciences, 16th Edition, Osol, A. Ed. 1980]. Acceptable carriers, excipients, or stabilizers are nontoxic to recipients at the dosages and concentrations employed, and include: buffers, such as phosphate, citrate, and other non-toxic organic acids; antioxidants such ascorbic acid; low molecular weight (less than 10 residues) polypeptides; proteins such as serum albumin, gelatin or immunoglobulins; hydrophilic polymers such polyvinylpyrrolidone; amino acids such as glycine, glutamine, asparagine, arginine, or lysine; monosaccharides, disaccharides, and other carbohydrates including glucose, mannose, or dextrans; chelating agents such as EDTA; sugar alcohols such as mannitol, or sorbitol; salt-forming counterions such as sodium; and/or nonionic surfactants such as Tween, Pluronics, or PEG.

The RIT or RMIT can be administered orally or by intravenous infusion, or they can be injected subcutaneously, intramuscularly, intraperitoneally, intrarectally, intravaginally, intranasally, intragastrically, intratracheally, intrapulmonarily, intratumorally, or intralesionally. They are preferably delivered directly to an appropriate tissue, e.g., a tumor or tumor bed following surgical excision of the tumor in order to kill any remaining tumor cells. Alternatively, they can be delivered to lymphoid tissue such as spleen, lymph nodes, or gut-associated lymphoid tissue in which an immune response (as, for example, in GVHD or an autoimmune disease) is occurring. The dosage required depends on the choice of the route of administration, the nature of the formulation, the nature of the patient's illness, the subject's size, weight, surface area, age, and sex, other drugs being administered, and the judgment of the attending physician. Suitable dosages are in the range of 0.01-100.0 μg/kg. Where a single therapeutic dose is given, this dosage will generally be in the range of 10-300 mCi total. Where multiple administrations are given, the total amount administered can be up to 700 mCi. Wide variations in the needed dosage are to be expected in view of the variety of possible RIT or RMIT, the differing efficiencies of various routes of administration, and whether the RIT or RMIT is being administered for therapeutic or imaging purposes. For example, oral administration would be expected to require higher dosages than administration by i.v. injection. Variations in these dosage levels can be adjusted using standard empirical routines for optimization as is well understood in the art. Administrations can be single or multiple (e.g., 2- or 3-, 4-, 6-, 8-, 10-, 20-, 50-, 100-, 150-, or more fold). Encapsulation of the polypeptide in a suitable delivery vehicle (e.g., polymeric microparticles or implantable devices) may increase the efficiency of delivery, particularly for oral delivery.

The following examples are meant to illustrate, not limit, the invention.

### EXAMPLES

### Example 1

### Generation on an IT for the Production of an RIT

Construction of a hybrid recombinant DTe23 encoding cDNA sequence. Construction of a hybrid recombinant cDNA sequence encoding an IT protein was achieved through “Splicing by Overlap Extension” (“SOE”). The hybrid recombinant cDNA sequence encoded, 5′ to 3′, a “start” methionine residue, the first 389 amino acids of diphtheria toxin (DT) [see co-pending U.S. application no. 09/440,344 which is incorporated herein by reference in its entirety], a flexible linker with the amino acid sequence EASGGPE (SEQ ID NO: 3), and a sFv antibody fragment derived from a monoclonal antibody (e23) specific for erbB2 (Her-2/neu) [Kasprzyk et al. (1992) Cancer Res. 52: 27721-2776]. The sFv fragment contained a flexible linker between the VH and the VL of a single GGGS unit. The hybrid recombinant cDNA sequence was generated as follows.

An NcoI restriction site containing a start codon (ATG) was attached at the 5′ end of the DT encoding sequence and a sequence encoding the flexible linker was attached to its 3′ end by PCR.

The cDNA sequence encoding the sFv specific for erbB2 was modified by PCR using the following two oligonucleotides as primers.

Forward: 5′-GAA GCT TCC GGA GGT CCC GAG GAC GTC CAG CTG ACC CAG-3′ (SEQ ID NO: 4)

Reverse: 5′-ACA CTC GAG TTA GGA GAC GGT GAC CGT GGT-3′ (SEQ ID NO: 5)

This PCR strategy added a sequence encoding the flexible linker mentioned above to the 5′ end of the erbB2 encoding sequence and a stop codon and a XhoI restriction site to its 3′ end. SOE was then used to generate the DTe23 encoding sequence which was ligated into the NcoI/XhoI site of the expression vector pET21-d (Novagen, Madison, Wis.) to give pET21-d.DTe23.

Expression of DTe23 fusion protein. The pET21-d.DTe23 plasmid was used to transform competent BL21 (DE3) E.coli cells (Novagen, Madison, Wis.). Briefly, four 100 ul aliquots of competent cells (as supplied by the manufacturer) were transformed with 1-2 ul of plasmid DNA. The cells were incubated on ice for 30 minutes and then heat shocked for 30 seconds at 42° C. After cooling on ice for 2 minutes, 1 ml of SOC medium was added to each aliquot and shaken at 37° C. for 1 hour. Each aliquot (about 100 ul) of cells was then plated onto an LB-agar bacterial culture plate containing carbenicillin (100 μg/ml). The plates were incubated at 37° C. overnight. The resulting lawn of cells was scraped from each plate and each cell population was resuspended in 1 liter of Superbroth supplemented with 100 μg/ml carbenicillin, 0.5% glucose, and 1.6 mM MgSO4. The culture was shaken at 37° C. until the A₆₀₀ was about 0.4-0.5. Protein expression was induced by adding isopropyl-β-D-thiogalactopyranoside (IPTG) (Gibco BRL, Gaithersburg, Md.) to each culture at a final concentration of 1 mM. After 90 minutes, the cells in each culture were pelleted by centrifugation at 4800 G, 4° C. for 10 minutes, thus resulting in 4 approximately equal bacterial pellet aliquots. The pellets were stored at -80° C.

Purification of the DTe23 fusion protein. Purification of DTe23 required isolation of the inclusion bodies from the bacterial pellets, solubilization of the inclusion bodies, and refolding followed by final purification of the protein by FPLC and HPLC column chromatography. Each bacterial pellet aliquot was resupended in 150 ml TE buffer (50 mM Tris, 20 mM EDTA, 100 mM NaCl, pH 8.0) and homgenized with a Tissuemizer (IKA LABORTECHNIK, Germany) intermittently for 45 seconds. Lysozyme (32 mg) was added to each aliquot and the suspensions were incubated at room temperature for 1 hour with intermittent shaking. Inclusion bodies were pelleted by centrifugation for 50 minutes at 4° C. at 24,000 G and the pellets were homogenized with a Tissuemizer four times with a centrifugation step followed by the addition of 20 ml fresh Triton X-100 buffer (11% v/v Triton X-100, 89% v/v TE buffer) between each homogenization. This procedure was repeated another 4 times in TE buffer without Triton X-100. Pelleted inclusion bodies were resuspended in 10 ml solubilization buffer (7 M guanidine-HCl, 0.1 M Tris, 2 mM EDTA, pH 8.0) and gently mixed for 30 minutes. The solution was sonicated on ice (3 ×30 minutes, 1 second pulse, 50% duty time). Protein concentration was measured by the Bradford Assay (typically 150-200 mg) and concentration was adjusted to 10 mg/ml. DTE (Dithioerythritol; Sigma, St. Louis, Mo.) was added to a final concentration of 10 mg/ml. The solution was gently mixed for 30 minutes then incubated overnight at room temperature. Refolding buffer (0.1 M Tris, 0.5 M L-arginine HCl, 0.9 mM glutathione, 2 mM EDTA, pH 8.0) in 100 fold excess of solubilized protein volume was made and stored at 10° C. The following morning the protein solution was centrifuged at 39,000 G for 10 minutes at room temperature. The supernatant was carefully separated from pelleted insoluble material and rapidly added dropwise to the refolding buffer while stirring. The resulting solution was incubated at 10° C for about 48 hours. The refolded protein solution was filtered with a 0.45 μm filter and diluted 10 fold in Milli-Q H₂O (prechilled to 4° C.) to a final volume of not greater than 20 liters. The sample was loaded overnight onto a Sepharose Q FPLC column (Pharmacia). Elution was achieved with a salt step gradient of NaCl in 20 mM Tris, pH 7.8. Steps of 20%, 30% and 100% NaCl were used. The DTe23 protein eluted in the 20% step. Fractions containing the protein were pooled and concentrated using a Centriprep-30 concentrator (Amicon, Beverly, Mass.) to a volume of 10 ml or less. The sample was then dialyzed against 2 changes of 4 liters of PBS using SpectraPor 2 dialysis tubing (molecular weight cut-off of 12-14 kDa; Spectrum, Laguna Hills CA) at 4° C. over 24 hours. The sample was then filtered with a 0.2 μm syringe filter and loaded onto a TosoHaas TSK-gel Column(cat#05147, G3000SW) which was eluted with PBS at a flow rate of 3 ml/min (generally 5 ml containing up to 10 mg protein). A first peak eluted from the column generally contained high molecular weight aggregated protein and was discarded. The second peak contained the monomeric DTe23. Fractions containing the monomeric DTe23 protein were pooled and concentrated with a Centriprep-30 concentrator to a final concentration of approximately 1 mg/ml. Purified protein was aliquoted and stored at −80° C.

### Example 2

### Cytotoxicity and Biodistribution Studies with an RIT

The DTe23 sFv anti-erbB2 (Her-2/neu) IT described above was labeled with ¹²⁵I using the Iodogen technique [Fraker et al. (1878) Biochem. Biophys. Res. Comm. 80:849-857] or with ^{99m}Tc. In brief, the Iodogen technique involved incubating 50 μg of DTe23 in 200 μl of 0.2 M phosphate buffer with Iodogen coated beads (Pierce Chemical Co.) and 0.5 or 1.0 mCi ¹²⁵I sodium iodide for 5 min. at room temperature. The labeled DTe23 was purified on a Dowex 1×8 column using Dulbecco's phosphate buffered saline (PBS). The final product contained 200-570 μCi ¹²⁵I linked to 50 μg of DTe23 (specific activity of 4.01-11.4 μCi per μg of protein) in a volume of 1.5 to 3.0 ml. For labeling with ^{99m}Tc, the DTe23 immunotoxin (220 μg) was mixed with 220 μL of acetate buffer (pH 5). The solution of ^{99m}Tc, containing stannous chloride and prereduced for 1 hour, was added and the mix was incubated at 45° C. for about 1 hour. Binding of ^{99m}Tc by this method does not involve the use of a chelating agent; the ^{99m}Tc atoms bind directly to the DTe23 molecules via free sulfhydryl groups on the DTe23 molecules.

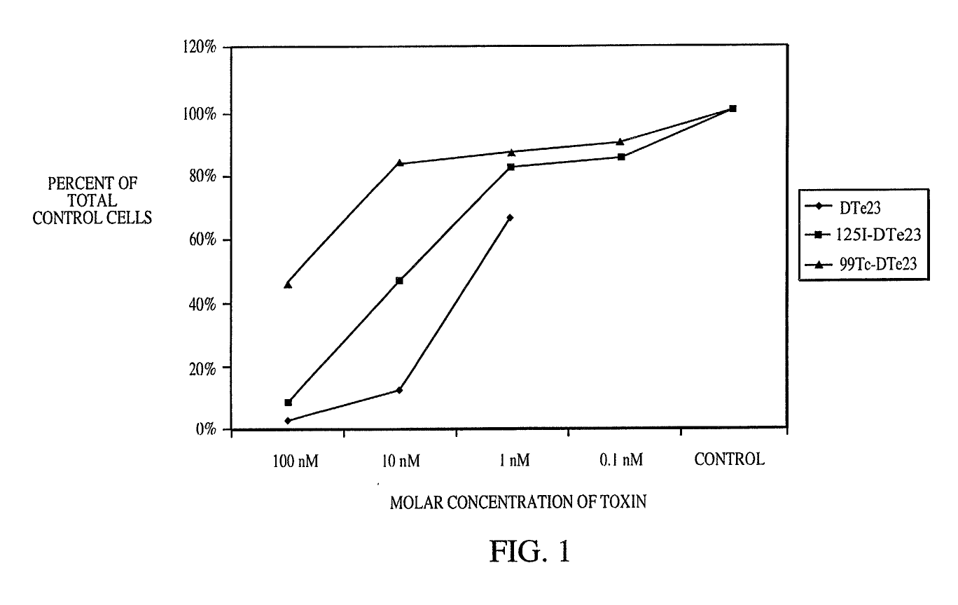

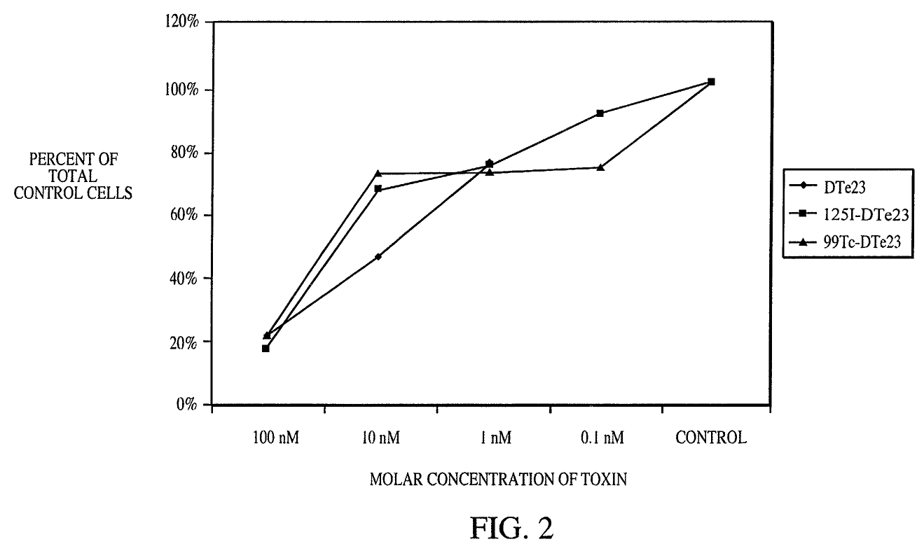

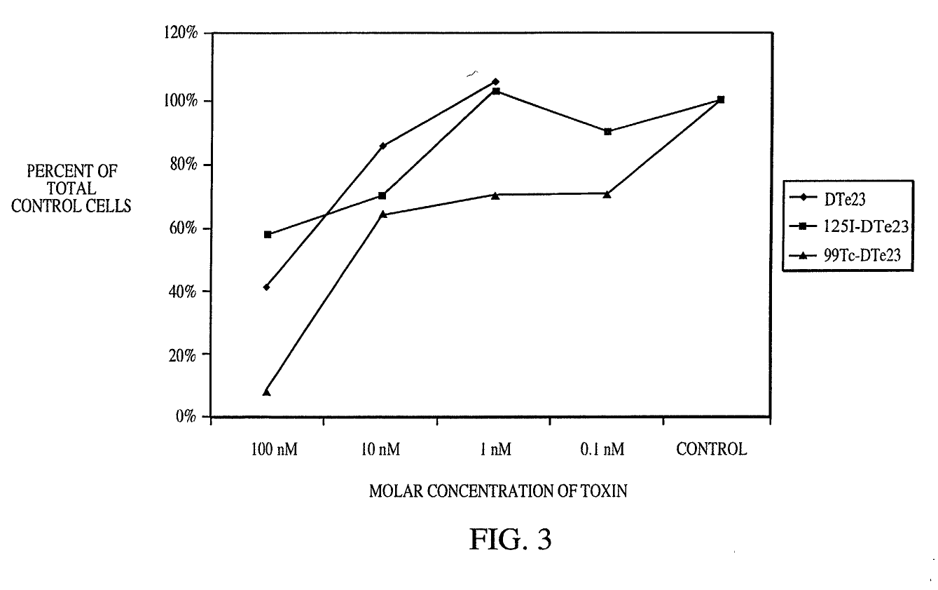

FIGS. 1, 2, and 3 show the in vitro cytotoxic activity of unlabeled and radiolabeled DTe23 against human breast (BT-474), ovarian (SKOV3.ip1), and colon (LS174T) cancer cells, respectively. In all cases, 2 ×10⁴ cells were plated into the wells of 24-well tissue culture plates and were incubated with the indicated immunotoxins at the indicated concentrations for 72 h. Remaining cells were detached from the tissue culture well bottoms by mild trypsin treatment and the number of viable cells was assessed by trypan blue dye exclusion or by means of a Coulter Counter. Data, which are means of triplicates, are presented in terms of the number of viable cells as a percentage of those in control wells containing neither an immunotoxic molecule nor the buffer used as a solvent for the relevant immunotoxic molecule. The data points labeled “control” on the x-axis of the graphs were obtained by calculating the number of the remaining cells in wells containing, instead of an immunotoxic molecule, buffer used as a solvent for the relevant immunotoxic molecule as a percentage of the number of viable cells in wells containing neither an immunotoxic molecule nor the buffer used as a solvent for the relevant immunotoxic molecule. The radiolabeled (^{99m}Tc and ¹²⁵I) DTe23 retained significant cytotoxic activity against BT-474 cells (FIG. 1) and SKOV3.ip1cells (FIG. 2) compared to unlabeled DTe23 and showed higher cytotoxicity against LS174T cells than the unlabeled DTe23 (FIG. 3).

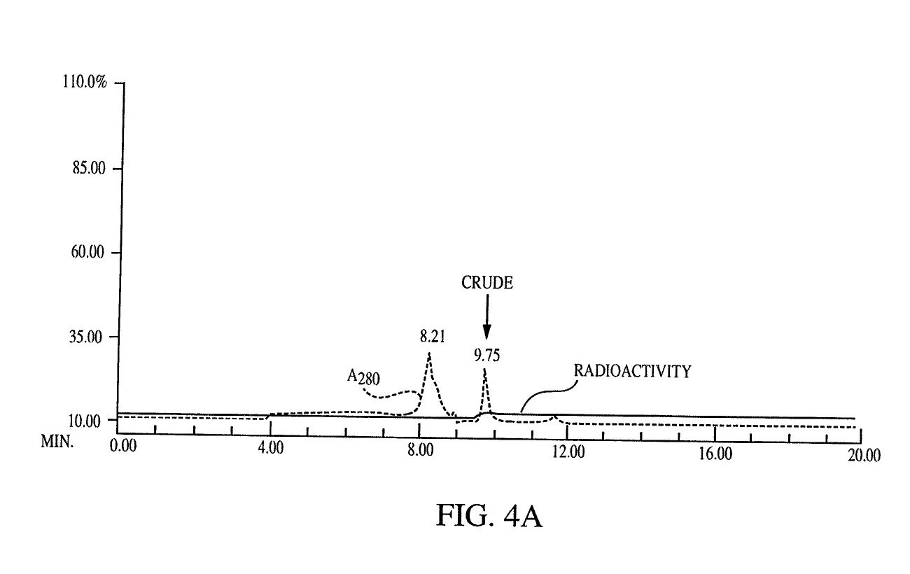

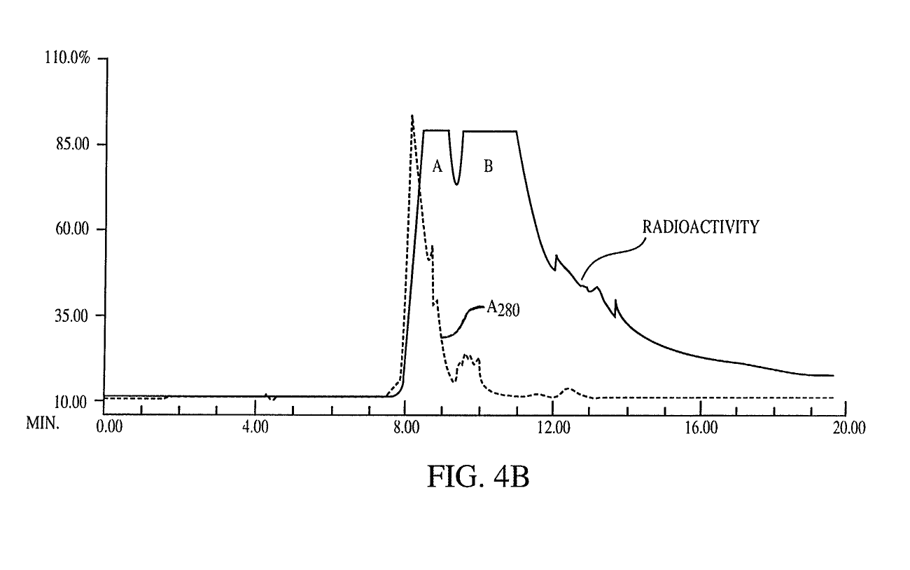

The DTe23 fusion protein was also labeled with ¹⁸⁸Re. DTe23 IT polypeptide (220 μg) was mixed with 220 μL of acetate buffer (pH 5). The solution of ¹⁸⁸Re, containing stannous chloride and prereduced for 1 h, was added and the mix was incubated at 45° C. for about 1 hour. As for ^{99m}Tc, binding of ¹⁸⁸Re by this method does not involve the use of a chelating agent; the ¹⁸⁸Re atoms bind directly to the DTe23 molecules via free sulfhydryl groups. Two sequential preparative high pressure liquid chromatography (HPLC) separations, starting with the radiolabeling reaction mixture in which the IT (DTe23) protein was labeled, were performed. In the first HPLC separation (FIG. 4A), material (“CRUDE”) corresponding to a protein (A₂₈₀) peak with a retention time of 9.75 minutes was collected. Some of this material was kept and used as semi-purified material (“crude prep”) in cytotoxicity and biodistribution experiments (see below) and the rest was subjected to a second HPLC separation (FIG. 4B). Material corresponding to radioactivity peak “A” in FIG. 4B was collected and used as a source of purified ¹⁸⁸Re labeled DTe23 (“fraction A”) in the cytotoxicity and biodistribution experiments below.

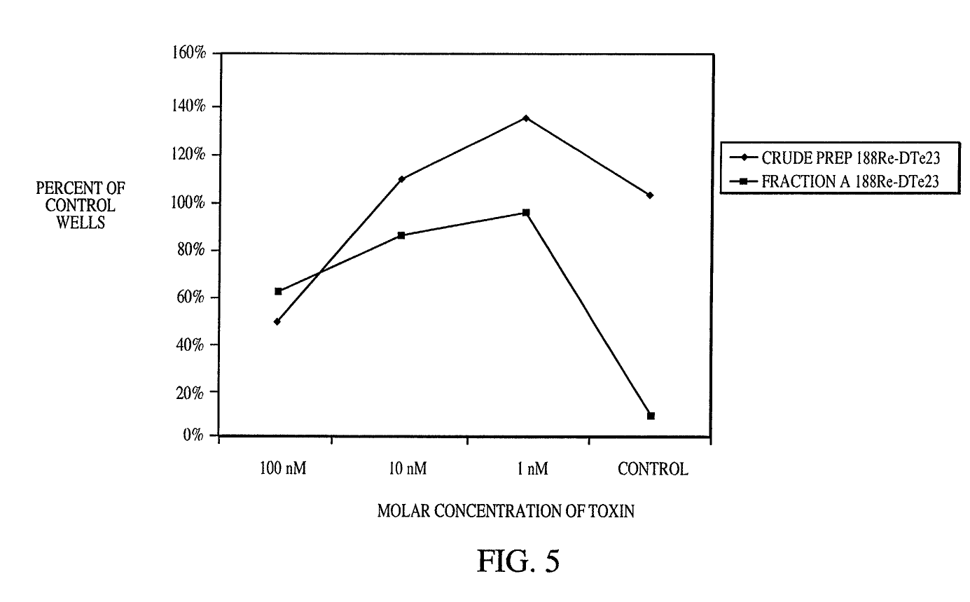

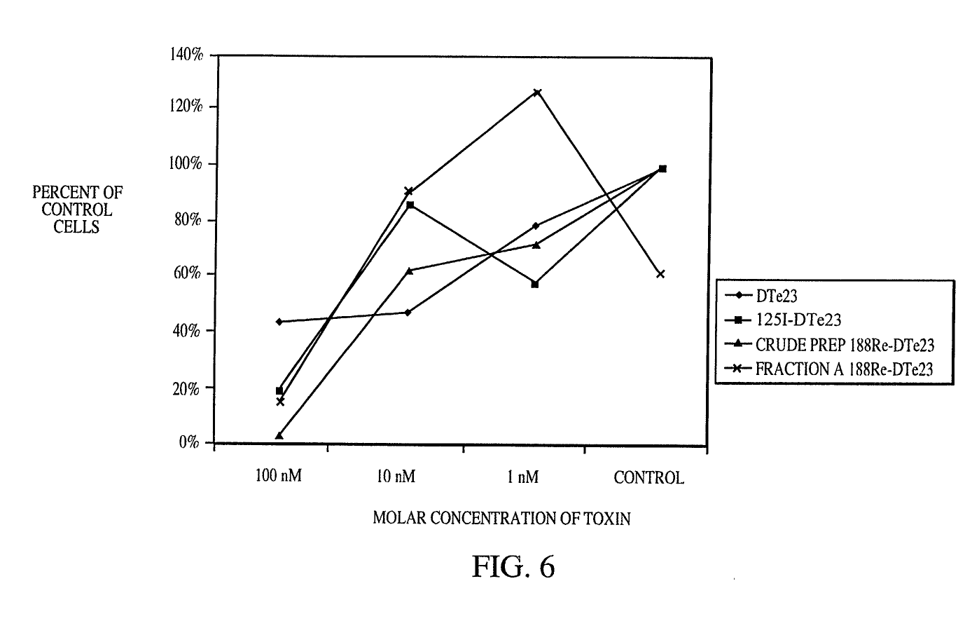

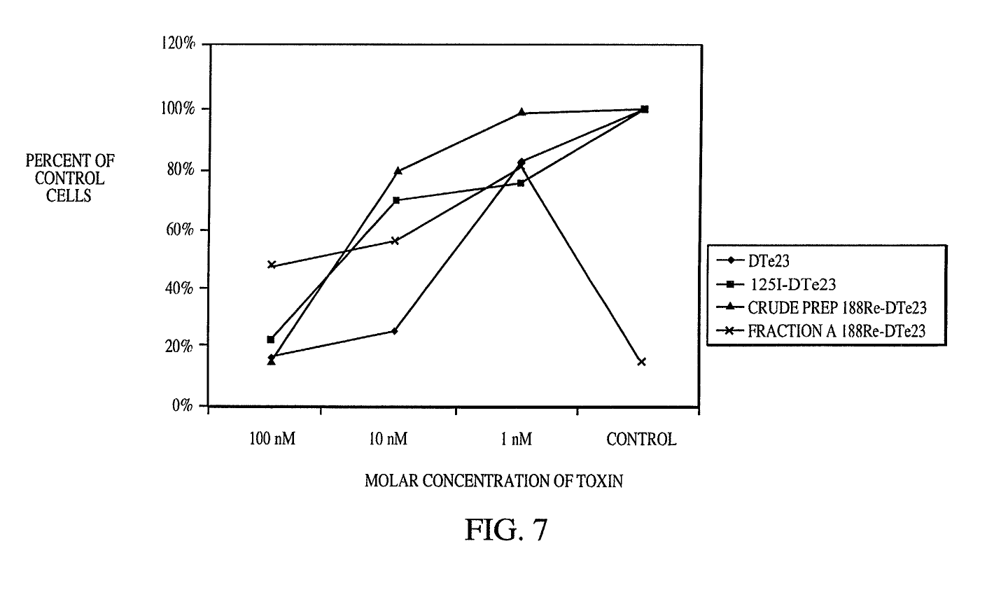

The results of in vitro cytotoxicity assays with ¹⁸⁸Re labeled DTe23 against breast (BT-474) colon (LS174T), and ovarian (SKOV3.ip1) cancer cells are shown in FIGS. 5, 6, and 7, respectively. The experiments were performed and the data were computed as in the experiments described above. The control value for fraction A was less than 100% in all three experiments because the HPLC buffer added to relevant culture wells was toxic to the cells. There was retention of cytotoxic activity of the ¹⁸⁸Re labeled DTe23 against all three cell lines. ¹²⁵I labeled DTe23 showed cytotoxicity against LS174T and SKOV3.ip1 cells.

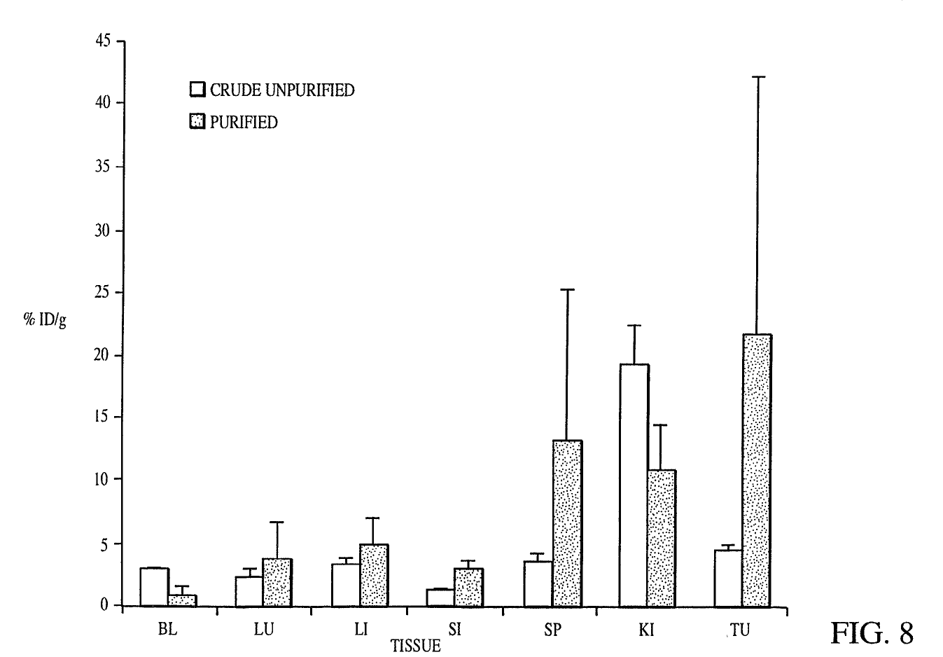

The results of a biodistribution study with ¹⁸⁸Re labeled DTe23 in athymic nude mice (n=3 mice per group) bearing intraperitoneal xenografts of LS174T human colon cancer cells are shown in FIG. 8. Means ± standard deviations are shown. The mice received an intraperitoneal injection of 5 ×10⁷ LS174T cells, and 11 days later received an intraperitoneal injection of 2 μCi ¹⁸⁸Re labeled DTe23 (corresponding to about 1.6 μg), either in the form of the crude reaction mixture (“Crude unpurified”) or peak A purified material (“Purified”). The indicated tissues or tumor were removed from the mice, weighed, and counted for radioactivity (γ radiation). The data are expressed as the percent of the injected dose per gram of tissue (“%ID/g”). There was evidence of localization of the ¹⁸⁸Re labeled DTe23 (crude mixture) in the tumor and clearance through the kidney. The purified material (peak A) showed increased localization in the tumor with a lower level of uptake in the kidney than the unpurified material.

### Example 3

### Determination of the In Vivo Maximum Tolerated Dose of an IT

The maximum tolerated dose of unlabeled DTe23 was determined in athymic nude mice. The animals received 1, 2.5, 5, or 10 μg DTe23 twice a day by intraperitoneal injection for 5 days. Survival of the animals was monitored. All 5 animals in the 1 and 2.5 μg per injection groups survived, whereas 5/5 animals (100%) in both the 5 and 10 μg per injection groups died. Death occurred in 9/10 high dose treatment (5 and 10 μg per injection) groups 5-7 days after the initiation of DTe23 injections.

### Example 4

### In vivo therapeutic activity of 2 RITs

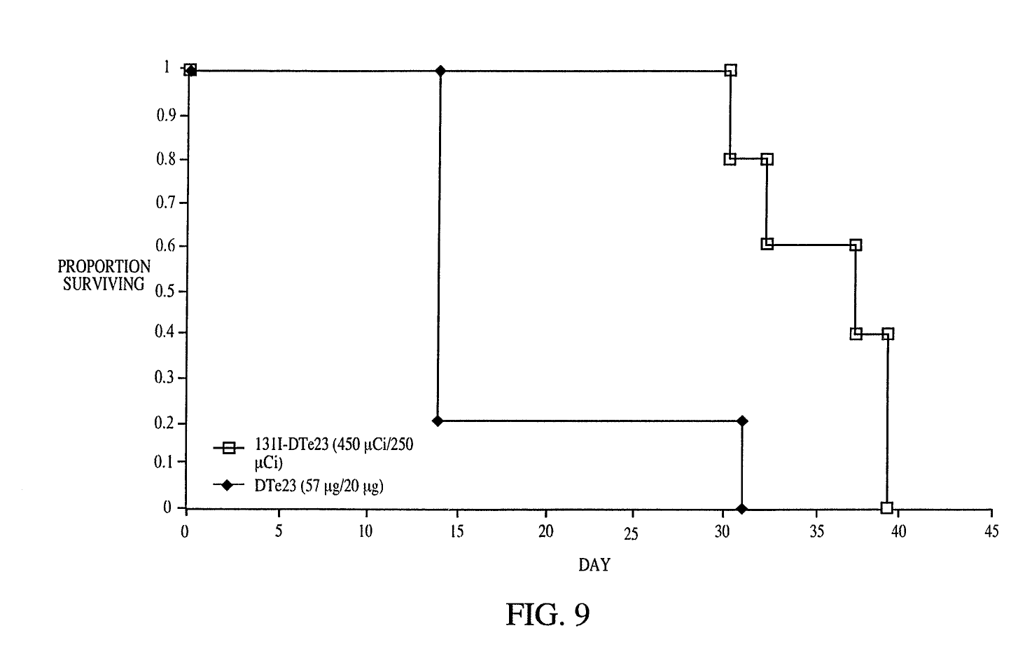

DTe23 was labeled with ¹³¹I to make ¹³¹I-DTe23 using the Iodogen technique as described in Example 2 for labeling of DTe23 with ¹²⁵I. Two groups of athymic nude mice (n=5 mice per group) were injected intraperitoneally (i.p.) with 3×10⁷ SKOV3.ip1 human ovarian cancer cells on day 0. All five mice in the first group were injected i.p. with unlabeled DTe23 (57 μg and 20 μg on days 7 and 9, respectively). All five mice in the second group were injected i.p. with ¹³¹I-DTe23 (450 μCi (57 μg) and 250 μCi (20 μg) on days 7 and 9, respectively). ¹³¹I-DTe23 was more effective than unlabeled DTe23 in prolonging the survival of mice with SKOV3.ip1 tumors (FIG. 9).

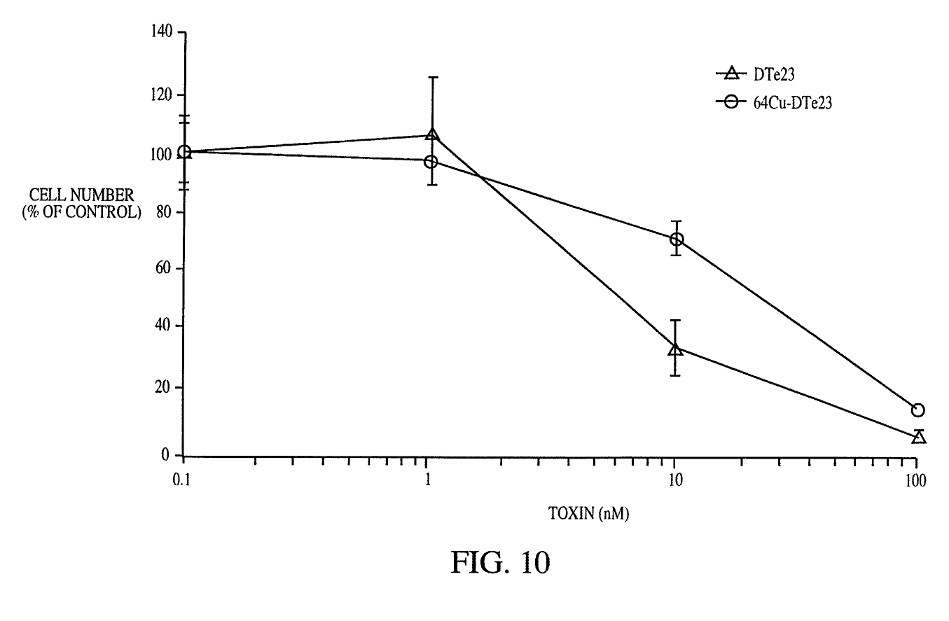

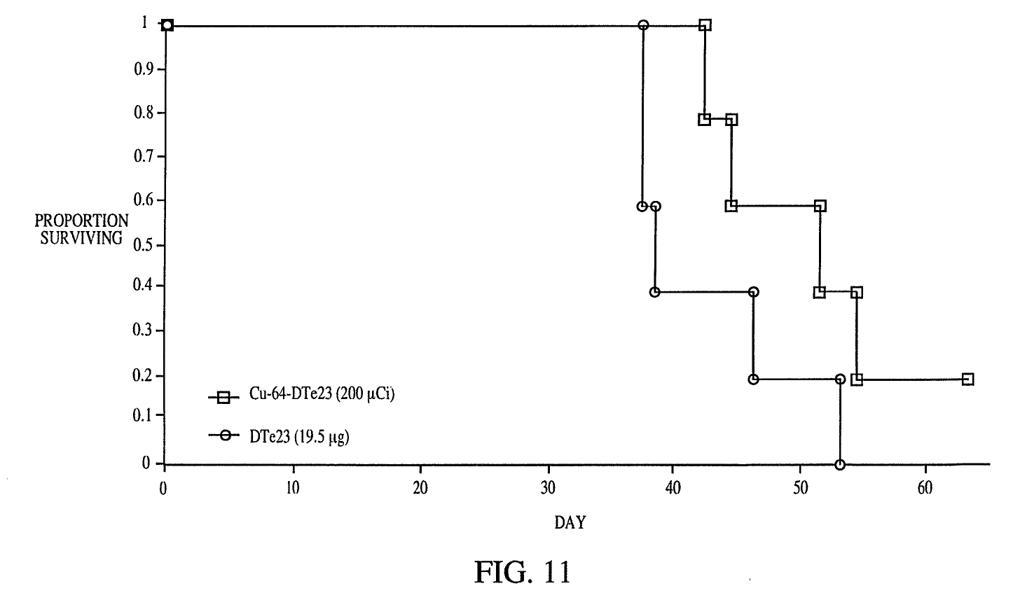

DTe23 was conjugated to the bifunctional chelating agent trisuccin by the keto-hydrazide method (Safavy et al. (1999) Bioconjug Chem 10:18-23). Briefly, 6-oxoheptanoic acid (OHA) was first conjugated to the immunotoxin to produce the OHA-DTe23 adduct which was purified by size-exclusion (SE) membrane filtration. The OHA-DTe23 adduct was then conjugated to trisuccin hydrazide at pH 5.5 for 18 h and the conjugate (trisuccin-DTe23) was purified by dialysis. The trisuccin-DTe23 conjugate (430 μg) was mixed with ⁶⁴Cu-copper acetate (8 mCi) and the solution was incubated at 35° C. for 1 h to produce ⁶⁴Cu-trisuccin-DTe23. This labeled conjugate was purified by SE chromatography to yield a solution containing approximately 350 μg of ⁶⁴Cu-trisuccin-DTe23 with a specific activity of 4 μCi/μg. The ⁶⁴Cu-trisuccin-DTe23 retained cytotoxic activity (compared to unlabeled DTe23) when tested against BT-474 human breast cancer cells in vitro essentially as described in Example 2 (FIG. 10). Two groups of athymic nude mice (n=5 mice per group) were injected i.p. with 5×10⁷ LS174T human colon cancer cells on day 0. All five in the first group were injected i.p. with DTe23 (19.5 μg) on day 4. All five mice in the second group were injected i.p. with 200 μCi (19.5 μg) ⁶⁴Cu-trisuccin-DTe23 on day 4. The radiolabeled immunotoxin had greater therapeutic efficacy than unlabeled immunotoxin (80% vs. 40% survival at 40 days after tumor cell injection, 20% vs. 0% survival at 60 days) (FIG. 11).

The above results indicate that radiolabeled immunotoxins are more effective than unlabeled immunotoxins at inhibiting tumor growth in vivo.

Although the invention has been described with reference to the presently preferred embodiment, it should be understood that various modifications can be made without departing from the spirit of the invention.

Accordingly, the invention is limited only by the following claims.

What is claimed is:

1. A radiolabeled immunotoxin comprising a toxic domain, a targeting domain, and at least one radionuclide atom, wherein the targeting domain is a single-chain Fv (sFv) antibody fragment that binds to a target molecule on a target cell, wherein the target molecule is not an ε chain of a T cell CD3 complex.

2. The radiolabeled immunotoxin of claim 1, wherein the toxic domain is a toxic polypeptide selected from the group consisting of: (a) ricin, (b) Pseudomonas exotoxin (PE); (c) bryodin; (d) gelonin; (e) α-sarcin; (f) aspergillin; (g) restrictocin; (h) angiogenin; (i) saporin; (j) abrin; (k)pokeweed antiviral protein (PAP); (l) a ribonuclease; (m) a pro-apoptotic polypeptide; and (n) a functional fragment of any of (a)-(m).

3. The radiolabeled immunotoxin of claim 1, wherein the toxic domain is diphtheria toxin (DT) or a functional fragment thereof.

4. The radiolabeled immunotoxin of claim 3, wherein the toxic domain comprises amino acids 1-389 of DT.

5. The radiolabeled immunotoxin of claim 1, wherein the target cell is a cancer cell.

6. The radiolabeled immunotoxin of claim 5, wherein the cancer cell is selected from the group consisting of a neural tissue cancer cell, a melanoma cell, a breast cancer cell, a lung cancer cell, a gastrointestinal cancer cell, an ovarian cancer cell, a testicular cancer cell, a lung cancer cell, a prostate cancer cell, a cervical cancer cell, a bladder cancer cell, a vaginal cancer cell, a liver cancer cell, a renal cancer cell, a bone cancer cell, and a vascular tissue cancer cell.

7. The radiolabeled immunotoxin of claim 5, wherein the target molecule is Her-2/neu.

8. The radiolabeled immunotoxin of claim 5, wherein the target molecule is selected from the group consisting of a mucin molecule, carcinoembryonic antigen (CEA), prostate-specific antigen (PSA), folate binding receptor, A33 alpha fetoprotein, CA-125 glycoprotein, colon-specific antigen p, ferritin, p-glycoprotein, G250, OA3, PEM glycoprotein, L6 antigen, 19-9, P97, placental alkaline phosphatase, 7E11-C5, 17-1A, TAG-72, 40 kDa glycoprotein, URO-8, a tyrosinase, an interleukin- (IL-)2 receptor polypeptide, an IL-3 receptor polypeptide, an IL-13 receptor polypeptide, an IL-4 receptor polypeptide, a vascular endothelial growth factor (VEGF) receptor, a granulocyte macrophage-colony stimulating factor (GM-CSF) receptor polypeptide, an epidermal growth factor (EGF) receptor polypeptide, an insulin receptor polypeptide, an insulin-like growth factor receptor polypeptide, transferrin receptor, estrogen receptor, a T cell receptor (TCR) α-chain, a TCR β-chain, a CD4 polypeptide, a CD8 polypeptide, a CD7 polypeptide, a B cell immunoglobulin (Ig) heavy chain, a B cell Ig light chain, a CD19 polypeptide, a CD20 polypeptide, a CD22 polypeptide, a MAGE polypeptide, a BAGE polypeptide, a GAGE polypeptide, a RAGE polypeptide, a PRAME polypeptide, and a GnTV polypeptide.

9. The radiolabeled immunotoxin of claim 1, wherein the radionuclide is selected from the group consisting of ⁹⁰Y, ¹⁸⁶Re, ¹⁸⁸Re, ⁶⁴Cu, ⁶⁷Cu, ²¹²Pb, ²¹²Bi, ²¹³Bi, ¹²³I, ¹²⁵I, ¹³¹I, ²¹¹At, ³²P, ¹⁷⁷Lu, ⁴⁷Sc, ¹⁰⁵Rh, ¹⁰⁹Pd, ¹⁵³Sm, ¹⁹⁹Au, ^{99m}Tc, ¹¹¹In, ¹²⁴I, ¹⁸F, ¹¹C, ¹⁹⁸Au, ⁷⁵Br, ⁷⁶Br, ⁷⁷Br, ¹³N, ^{34m}Cl, ³⁸Cl, ^{52m}Mn, ⁵⁵Co, ⁶²Cu, ⁶⁸Ga, ⁷²As, ⁷⁶As, ⁷²Se, ⁷³Se, and ⁷⁵Se.

10. A radiolabeled multimeric immunotoxin comprising:
(a) at least two monomers; and
(b) at least one radionuclide atom,
wherein each monomer comprises a targeting domain and a toxic domain and is physically associated with the other monomers,
wherein the targeting domain binds to a target molecule on a target cell.

11. The radiolabeled multimeric immunotoxin of claim 10, wherein each of said monomers further comprises one or more coupling moieties and the physical association of the monomer is by at least one of the one or more coupling moieties.

12. The radiolabeled multimeric immunotoxin of claim 11, wherein the coupling moiety is a terminal moiety.

13. The radiolabeled multimeric immunotoxin of claim 12, wherein the terminal moiety is a C-terminal moiety.

14. The radiolabeled multimeric immunotoxin of claim 11, wherein the one or more coupling moieties are cysteine residues.

15. The radiolabeled multimeric immunotoxin of claim 11, wherein at least one of the one or more coupling moieties is a heterologous coupling moiety.

16. The radiolabeled multimeric immunotoxin of claim 10, wherein each of the monomers comprises the same amino acid sequence.

17. An in vitro method of killing a target cell, the method comprising culturing the target cell with the radiolabeled immunotoxin of claim 1.

18. A method comprising:
(a) identifying a subject suspected of having a pathogenic cell disease; and
(b) administering to the subject a radiolabeled immunotoxin comprising a toxic domain, a targeting domain, and at least one radionuclide atom, wherein the targeting domain is a sFv antibody fragment that binds to a target molecule on a target cell in the subject.

19. The method of claim 18, wherein the toxic domain is a toxic polypeptide selected from the group consisting of: (a) ricin, (b) Pseudomonas exotoxin (PE); (c) bryodin; (d) gelonin; (e) α-sarcin; (f) aspergillin; (g) restrictocin; (h) angiogenin; (i) saporin; (j) abrin; (k) pokeweed antiviral protein (PAP); (l) a ribonuclease; (m) a pro-apoptotic polypeptide, and (n) a functional fragment of any of (a)-(m).

20. The method of claim 18, wherein the toxic domain is diphtheria toxin (DT) or a functional fragment thereof.

21. The method of claim 20, wherein the functional fragment comprises amino acids 1-389 of DT.

22. The method of claim 18, wherein the target cell is a cancer cell.

23. The method of claim 22, wherein the cancer cell is selected from the group consisting of a neural tissue cancer cell, a melanoma cell, a breast cancer cell, a lung cancer cell, a gastrointestinal cancer cell, an ovarian cancer cell, a testicular cancer cell, a lung cancer cell, a prostate cancer cell, a cervical cancer cell, a bladder cancer cell, a vaginal cancer cell, a liver cancer cell, a renal cancer cell, a bone cancer cell, and a vascular tissue cancer cell.

24. The method of claim 22, wherein the target molecule is Her-2/neu.

25. The method of claim 22, wherein the target molecule is selected from the group consisting of a mucin molecule, CEA, PSA, folate binding receptor, A33 alpha fetoprotein, CA-125 glycoprotein, colon-specific antigen p, ferritin, p-glycoprotein, G250, OA3, PEM glycoprotein, L6 antigen, 19-9, P97, placental alkaline phosphatase, 7E11-C5, 17-1A, TAG-72, 40 kDa glycoprotein, URO-8, a tyrosinase, an interleukin(IL-)2 receptor polypeptide, an IL-3 receptor polypeptide, an IL-13 receptor polypeptide, an IL-4 receptor polypeptide, a VEGF receptor, a GM-CSF receptor polypeptide, an EGF receptor polypeptide, an insulin receptor polypeptide, an insulin-like growth factor receptor polypeptide, transferrin receptor, estrogen receptor, a T cell receptor (TCR) α-chain, a TCR β-chain, a CD4 polypeptide, a CD8 polypeptide, a CD7 polypeptide, a B cell Ig heavy chain, a B cell Ig light chain, a CD19 polypeptide, a CD20 polypeptide, a CD22 polypeptide, a MAGE polypeptide, a BAGE polypeptide, a GAGE polypeptide, a RAGE polypeptide, a PRAME polypeptide, and a GnTV polypeptide.

26. The method of claim 18, wherein the method is a method of killing a target cell in the subject.

27. The method of claim 26, wherein the radionuclide is selected from the group consisting of ⁹⁰Y, ¹⁸⁶Re, ¹⁸⁸Re, ⁶⁴Cu, ⁶⁷Cu, ²¹²Pb, ²¹²Bi, ²¹³Bi, ¹²³I, ¹²⁵I, ¹³¹I, ²¹¹At, ³²P, ¹⁷⁷Lu, ⁴⁷Su, ¹⁰⁵Rh, ¹⁰⁹Pd, ¹⁵³Sm, and ¹⁹⁹Au.

28. The method of claim 18, wherein the method is an imaging method.

29. The method of claim 28, wherein the radionuclide is selected from the group consisting of ¹⁸⁶Re, ¹⁸⁸Re, ⁶⁴Cu, ⁶⁷Cu, ²¹²Bi, ¹²³I, ¹³¹I, ²¹¹At, ¹⁷⁷Lu, ⁴⁷Sc, ¹⁰⁵Rh, ¹⁰⁹Pd, ¹⁵³Sm, ¹⁹⁹Au, ^{99m}Tc, ¹¹¹In, ¹²⁴I, ¹⁸F, ¹¹C, ¹⁹⁸Au, ⁷⁵Br, ⁷⁶Br, ⁷⁷Br, ¹³N, ^{34m}Cl, ³⁸Cl, ^{52m}Mn, ⁵⁵Co, ⁶²Cu, ⁶⁸Ga, ⁷²As, ⁷⁶As, ⁷²Se, ⁷³Se, and ⁷⁵Se.

30. A method of making a radiolabeled immunotoxin, the method comprising:
(a) providing a cell comprising a vector containing a nucleic acid sequence encoding a protein, the nucleic acid sequence being operably linked to a transcriptional regulatory element (TRE);
(b) culturing the cell;
(c) extracting the protein from the culture; and
(d) attaching at least one radionuclide atom to the protein,
wherein the protein comprises a toxic domain and a targeting domain,
wherein the targeting domain is a sFv antibody fragment that binds to a target molecule on a target cell, wherein the target molecule is not a polypeptide of the CD3 complex.

31. A method of making a radiolabeled multimeric immunotoxin, the method comprising:
(a) providing one or more cells, each of the cells comprising a nucleic acid sequence encoding a monomer with a different amino acid sequence, wherein the nucleic acid sequence is operably linked to a TRE;
(b) separately culturing each of the one or more cells;
(c) extracting the monomer from each of the cultures;
(d) exposing the monomers to conditions which allow multimerization of the monomers to form a multimer comprising at least two monomers; and
(e) attaching at least one radionuclide atom to the multimer,
wherein each monomer comprises a targeting domain and a toxic domain,
wherein the targeting domain binds to a target molecule on a target cell.

32. A method of making a radiolabeled immunotoxin, the method comprising:
(a) providing a protein comprising a toxic domain and a targeting domain; and
(b) attaching at least one radionuclide atom to the protein,
wherein the targeting domain is a sFv antibody fragment that binds to a target molecule on a target cell, wherein the target molecule is not an ε chain of a T cell CD3 complex.

33. A method of making a radiolabeled multimeric immunotoxin, the method comprising:
(a) providing a multimeric protein; and
(b) attaching at least one radionuclide atom to the multimeric protein;
wherein the multimeric protein comprises at least two monomers,
wherein each monomer comprises a targeting domain and a toxic domain and is physically associated with the other monomers,
wherein the targeting domain binds to a target molecule on a target cell.

34. The radiolabeled multimeric immunotoxin of claim 10, wherein the targeting domain is an antibody fragment.

35. The radiolabeled multimeric immunotoxin of claim 34, wherein the antibody fragment is a sFv.

36. The radiolabeled multimeric immunotoxin of claim 34, wherein the antibody fragment binds to a target molecule on a T cell.

37. The radiolabeled multimeric immunotoxin of claim 34, wherein the target molecule is a CD3 polypeptide.

38. The radiolabeled multimeric immunotoxin of claim 10, wherein the targeting domain is a targeting polypeptide selected from the group consisting of: (a) a cytokine; (b) a ligand for a cell adhesion receptor; (c) a ligand for a signal transduction receptor; (d) a hormone; (e) a molecule that binds to a death domain family molecule; (f) an antigen; and (g) a functional fragment of any of (a) - (f).

39. The radiolabeled immunotoxin of claim 1, further comprising one or more additional targeting domains.
